(19) United States
(12) Patent Application Publication
(10) Pub. No.: US 2023/0392101 A1
(43) Pub. Date: Dec. 7, 2023

(54) Photobioreactor, in particular for the production of microorganisms such as microalgae

(71) Applicant: Anita Meier, Weichering (DE)

(72) Inventor: Anita Meier, Weichering (DE)

(21) Appl. No.: 18/249,110

(22) PCT Filed: Oct. 13, 2021
(86) PCT No.: PCT/EP2021/078330

§ 371 Date: Apr. 14, 2023

(30) Foreign Application Priority Data
Oct. 14, 2020 (DE) 10 2020 127 005.4

(51) Int. Cl. C12M 1/00

(52) U.S. Cl. CPC C12M 21/02; C12M 23/34; C12M 31/10; C12M 23/58

(57) ABSTRACT

The invention relates to a photobioreactor (1), in particular for the production of microorganisms, wherein the photobioreactor (1) is designed as a closed reactor which has a plurality of upwardly open reactor containers (2) which are closed by a top wall (7) of the photobioreactor (1) and in which a nutrient medium can be accommodated. According to the invention, at least some of the reactor containers (2) are designed as individual containers, whereby adjacent reactor containers (2) form a gap (13) between a front wall (3) and a rear wall (4), which gap is closed at the top by an overflow wall region (12) and has a container overflow opening (16) between the adjacent reactor containers (2). At least one lighting element (29) is received in the gap (13). Furthermore, in each of the reactor containers (2) a partition (6) is provided which divides the reactor container (2) into a front reactor chamber (8) and a rear reactor chamber (9), wherein in the partition (6), in the bottom wall side region, at least one partition-through-flow opening (10) is provided between the front and rear reactor chambers (8, 9). At least one or at least a part of the reactor containers (2) has at least one feeding device (33) by means of which a CO2-containing medium can be introduced into at least one reactor container (2) from outside the reactor container (2), wherein the CO2-containing medium is a CO2-containing gas or a CO2 obtained from a CO2-containing gas. The invention further claims a reactor container and a method.

The invention refers to a photobioreactor, in particular for the production of microorganisms, according to the preamble of claim 1, a biogas plant with at least one photobioreactor according to the preamble of claim 29, a reactor for the gasification of fuels according to the preamble of claim 30, a reactor container for a photobioreactor according to the preamble of claim 31, and a process for the production of microorganisms, in particular microalgae, according to the preamble of claim 32.

Microalgae are prokaryotic and eukaryotic photosynthetic microorganisms characterized by simple cellular material. Depending on the species, microalgae can vary in size from a few micrometers to several hundred micrometers (μm). Microalgae can either live as single cells or form colonies. Depending on their size, microalgae can be divided into four main categories: Microplankton (20-1000 μm), Nanoplankton (2-100 μm), Ultraplankton (0.5-15 μm) and Picoplankton (0.2-2 μm). Important microalgae types for industrial production are, for example, Chlorella vulgaris, Spirulina (Arthrospira) and Nannochloropsis, to name just a few examples.

Due to their morphological and physiological properties, microalgae are used in various biotechnological processes, such as the production of antioxidants, pharmaceuticals, immunostimulants, biofuels, peptides, polymers, toxins, sterols and food supplements, to mention just a few examples. Depending on the microalgae species used, biomass cultivation and subsequent processing, valuable molecules and compounds such as fat, oil, polyunsaturated fatty acids, natural dyes, various polysaccharides, pigments, bioactive molecules, etc. can also be obtained from microalgae. Besides lipids, carbohydrates are also valuable raw materials of microalgae. Various studies indicate that microalgae proteins are of high value and comparable to conventional plant proteins. The simple cellular structure of microalgae also allows easier genetic manipulation compared to, for example, plants.

The most important criteria affecting the quality of the microalgal biomass produced are the selection of the microalgae, the selection of the appropriate bioreactor system, the selection of the optimal conditions for microalgal cultivation, and the selection of the process for separating the desired microbial product. Thus, the conditions of microalgae cultivation in a bio reactor and the selected bio reactor system have a great influence on the production of microalgae.

Biotechnological microalgae production takes place in open or closed bio reactor systems. Open systems are mostly natural watercourses, lakes and lagoons as well as artificially excavated channels and basins.

There are various constructive solutions for closed bio reactor systems relating to the subject matter of the present invention, such as bio reactors in the form of pipes or plates, in which the movement of the always liquid nutrient medium (often also referred to as growth medium or nutrient solution), in which the microalgae are formed, is carried out by using pumps.

These bio reactors, which can be used to produce microorganisms such as microalgae, i.e. to cultivate and propagate them, are often also referred to as photobioreactors, since they use carbon dioxide (CO2) and light to perform photosynthesis for the growth and propagation of the microorganisms in a known manner.

A closed photobioreactor for the recovery of phytoplankton is already known from EP 3 041 924 B2, in which a nutrient solution and several vertically aligned and horizontally spaced plates are present in a housing, which plates are not intended to extend as far as the opposite wall and at least some of which are attached to the top of the housing in order to form a vertically meandering flow. The plates are alternately attached either to the bottom or to the top of the housing, with illumination means attached to the face of the plates located in the area of attachment. The plates themselves consist of a so transparent solid material in which light-scattering particles are embedded with a particle density such that the density of the light emission is approximately constant over the surface of the plate. However, a large-scale production system of this kind is relatively complex in design and therefore expensive to manufacture.

Furthermore, a closed photobioreactor for the cultivation and reproduction of microorganisms is known from EP 2 326 706 B1, which has a tank system with a nutrient suspension, wherein the tank system has a vertical meander system formed by partition walls which are at least partially translucent, in order to achieve an essentially vertical flow of the nutrient suspension in the tank system. In a technically extremely complex manner, the partition walls here are hollow and filled with a dispersive liquid for conducting light into the nutrient suspension.

In contrast, it is an object of the present invention to provide a photobioreactor, in particular for the production of microorganisms, most preferably microalgae, which is of simple construction, which is also simple to maintain, and with which a high yield can be achieved in conjunction with a high-quality product. It is a further object of the present invention to provide a suitable reactor container for such a photobioreactor. And finally, it is a further task of the invention to provide a suitable method for the production of microorganisms, in particular microalgae with such a photobioreactor.

These tasks are solved by the features of the independent patent claims. Advantageous designs are the subject of the related subclaims.

According to claim 1, a photobioreactor, in particular for the production of microorganisms, most preferably of microalgae, is provided, wherein the photobioreactor is designed as a closed reactor comprising a plurality of upwardly open reactor containers which are closed by at least one or a single-part or multi-part, preferably removable, top wall of the photobioreactor (preferably closed in a gas-tight and/or liquid-tight manner) and in which a nutrient medium can be accommodated. The basic function of the top wall is that of a lid to reduce contamination of the nutrient medium or the produced microorganisms with impurities (for example solid particles from air, bacteria, spores, etc.), resulting in a high quality of the produced microorganisms. Preferably, the top wall can be opened at any time to facilitate access to the nutrient or growth medium and for cleaning the reactor container. A nutrient medium in this context means any suitable liquid growth medium inoculated with nutrients to initiate the production of the respective desired microorganisms. In the case of microalgae, for example, this can be osmosis water inoculated with nutrients.

According to the invention, at least some of the reactor containers, preferably all reactor containers, of the photobioreactor are designed as individual containers which, viewed in cross-section, each have a U-shape with a front wall extending in the direction of the vertical axis and a rear wall spaced apart therefrom in the longitudinal direction and likewise extending in the direction of the vertical axis, which are connected to one another at the bottom by a bottom wall. The reactor containers of the photobioreactor, which are designed as individual containers as described above, are arranged one behind the other as seen in the longitudinal direction of the photobioreactor (or the direction of flow of the nutrient medium) in such a way that a front reactor container, as seen in the longitudinal direction, is connected to a rear wall, which is transparent to light at least in some areas, with the formation of a gap or gap distance. gap distance adjoins an at least partially light-permeable front wall of a rear reactor container, as seen in the longitudinal direction, the free end regions of the front and rear walls adjoining one another with the formation of the gap having a common overflow wall region which closes the gap from above, with respect to the vertical axis direction, and which has at least one container overflow opening between the adjoining reactor containers. Via this container overflow opening, the nutrient medium can then flow from a front reactor container, as seen in the direction of flow, into a rear reactor container. The front wall and the rear wall of the reactor container(s) are preferably rectangular and/or plate-shaped.

The overflow wall region, which can also be referred to as the overflow wall region element, extends up to the top wall and adjoins it. This adjacency is preferably effected in such a way that the overflow wall region adjoins the top wall in a gas-tight and/or liquid-tight manner and/or, if necessary, is even connected to it (preferably detachably connected).

At least one lighting element is accommodated in the gap between adjacent reactor containers (and thus below the overflow wall region as viewed in the vertical axis direction), by means of which light can be emitted through the respectively assigned front and/or rear wall, which is designed to be at least partially translucent, into one of the two adjacent reactor containers or into both adjacent reactor containers.

Furthermore, in each of the reactor containers designed as individual vessels, a partition is provided which is preferably connected to the bottom wall and/or is rectangular and/or plate-shaped and which, starting from the bottom wall, extends upwards in the vertical axis direction to the top wall and adjoins the latter, preferably adjoins the latter in a gas-tight and/or liquid-tight manner and/or is possibly even connected to the latter (preferably detachably connected), so that the partition divides the reactor container, in relation to the longitudinal direction or flow direction, into a front reactor chamber and a rear reactor chamber. direction of flow, into a front reactor chamber and a rear reactor chamber.

Further, in the partition, in the bottom wall side adjoining and/or connecting region of the partition to the bottom wall, at least one partition-through-flow opening is provided between the front and rear reactor chambers.

At least one or at least a part of the reactor containers further comprises at least one feeding device by means of which a CO2-containing medium can be introduced from outside the reactor container into at least one reactor container, wherein the CO2-containing medium is a CO2-containing gas or CO2 obtained from a CO2-containing gas.

With such a structure, a nutrient medium received in the front reactor chamber of a front reactor container can flow through the at least one partition-through-flow opening into the rear reactor chamber of the front reactor container and then further flow upwardly or through the at least one container overflow opening from the rear reactor chamber of the front reactor container into a front reactor chamber of a rear reactor container (vertical meandering flow).

The particular advantage of the solution according to the invention is that a plurality of essentially individual reactor containers, preferably formed as identical parts, can be provided here, which can be easily manufactured, for example also by 3D printing. The individual reactor containers can be joined together in basically any number and sequence to form a desired photobioreactor, whereby the joining together then takes place in such a way that in a gap or intermediate space between the respective rear wall and front wall of adjacent reactor containers the light-donating lighting elements required for photosynthesis can be arranged simultaneously in an advantageous dual function in a simple and functionally reliable manner. The gap between the adjacent reactor containers is easily accessible, especially for maintenance and assembly work, so that the lighting elements and their light sources can be easily replaced. A technically complex solution according to the state of the art, in which lighting elements are to be arranged on the front surface of plates into which light-scattering particles of a certain particle density are to be embedded in a particularly complex manner, can thus be completely dispensed with by the solution according to the invention, as can the provision of dispersive liquids in hollow partition walls, which is also known from the state of the art. With the solution according to the invention, the illuminants only have to be arranged at the desired height in the externally accessible gap between the front and rear walls of adjacent reactor containers.

This solution also has the advantage over the solutions of the prior art that the illumination of the reactor containers or the reactor chambers can be individually adapted and changed in a simple manner. For this purpose, it is sufficient, for example, to change the arrangement and orientation of the lighting elements, which can be done simply from outside the reactor container by intervening in the gap between the adjacent reactor containers. In addition, the respective lighting conditions inside the reactor containers can be easily specified, for example in such a way that differently bright areas are formed as seen in the direction of flow, which is advantageous for the growth of microorganisms, in particular microalgae, and will be explained in more detail below.

In addition, the solution according to the invention with the individual reactor containers also has the advantage that, in the event of damage to an individual reactor container or individual parts of a reactor container, only this individual reactor container has to be replaced.

In addition, this results in a particularly advantageous modular system, which can be supplemented or reduced by one or more reactor containers in a particularly simple manner, if this should be necessary in the practical operation of the photobioreactor.

A particularly advantageous connection between the adjacent reactor containers is provided by the common overflow wall region which closes the gap from above and which, as will be explained in more detail below, can, for example, be formed integrally with one or possibly even both of the adjacent reactor containers or, alternatively, can be formed by a separate component.

The feeding device, by means of which a CO2-containing medium can be introduced from outside the reactor container into at least one reactor container, the CO2-containing medium being a CO2-containing gas or a CO2 obtained from a CO2-containing gas, also has the effect that CO2 or a CO2-containing gas can be introduced selectively into the reactor container(s), which has a particularly beneficial effect on photosynthesis.

At this point, it should be expressly clarified once again that, in accordance with the present invention, it is indeed preferred that all reactor containers of the photobioreactor are designed as individual containers, as this has been described above and will also be described further below. However, the scope of protection also expressly includes those embodiments of a photobioreactor in which only some, that is, for example, at least two, of the reactor containers of the photobioreactor are designed as such individual containers and the rest of the reactor containers are constructed differently. The advantages according to the invention then arise only for the individual containers designed according to the invention and arranged one behind the other, but they are nevertheless present, so that such embodiments are also expressly included in the scope of protection of the solution according to the invention. This must always be observed in principle in all the following embodiments and further developments, even if this is no longer expressly repeated.

The term U-shape is always and explicitly to be understood in a broad sense here, as in the following, and refers above all to the U-shaped flow taking place in the individual containers. In this respect, a U-shaped container naturally also includes V-shaped and thus pointed container designs, in which the bottom wall is formed by the tip and the areas immediately adjoining it.

The CO2-containing gas can in principle be any CO2-containing waste gas produced during combustion, for example during the combustion of fuels, in particular waste gas from a power plant or an industrial plant. However, a CO2-containing biogas obtained by fermenting biomass in a biogas plant is particularly suitable as a CO2-containing gas. The biogas is produced there by a preferably anaerobic decomposition of organic material by microorganisms with the release of methane and—the product desired here—carbon dioxide (CO2), for example in at least one fermenter of the biogas plant. Biogas production essentially proceeds in four successive stages: In the hydrolysis phase, the solid substances (proteins, fats, carbohydrates) are broken down (hydrolyzed) by bacterial enzymes into simpler components (for example: amino acids, glucose, fatty acids), which are now water-soluble. In the subsequent second phase (acidification), the dissolved substances are further broken down into organic acids (acetic acid, propionic acid, butyric acid), lower alcohols, aldehydes, hydrogen, carbon dioxide and other gases such as ammonia and hydrogen sulfide. Acidification occurs very rapidly until the bacteria are inhibited in their degradation process by their own degradation products (low pH). In the third phase (acetogenic stage), the intermediates are further converted to acetic add. In the fourth and final stage (methane formation), the final products methane and carbon dioxide are formed from acetic acid, hydrogen and carbon dioxide in a strictly anaerobic environment, preferably at a pH of 6.7 to 8.0. Biogas usually consists of about 40 to 75% methane and about 25 to 60% carbon dioxide.

Alternatively or additionally, it can also be provided that the CO2-containing gas is a CO2-containing synthesis gas from a reactor for the gasification of fuels, in particular for the gasification of carbon-containing solid fuels (for example biomass and here in particular wood or similar materials). In particular, thereto-chemical biomass gasification is a process that aims to convert a biogenic solid fuel as completely as possible into a combustible gas under the action of heat. The biomass is reacted with a gasification agent (air, oxygen, water steam or carbon dioxide) that introduces bound or free oxygen into the process. Thermal cracking and partial oxidation produce a product gas which, depending on the feedstock, reaction conditions and gasification agent, consists mainly of varying concentrations of hydrogen (H2), carbon monoxide (CO), carbon dioxide (CO2), water steam (H2O), methane (CH4) and, in the case of gasification with air, nitrogen (N2). In addition to the product gas, charcoal or ash with different residual carbon contents and condensable low molecular weight hydrocarbons, collectively usually referred to as tars, are produced as products of incomplete gasification. The gasification process consists of 4 phases, namely drying, pyrolysis, oxidation and reduction. The proportion of CO2 in the synthesis gas is less than that in the biogas and is usually between 10 and 30%, also depending on the fuel and/or gasification agent selected.

According to a particularly preferred embodiment, a gas scrubber is provided by means of which the gas containing CO2 can be separated from the gas (biogas or synthesis gas) by means of a gas scrubber, preferably by chemical absorption. Specifically, the gas scrubber can have an absorber column for this purpose, for example a counter-current absorber column, in which the CO2 present in the gas is absorbed by a solvent. In addition, a stripping column is then preferably provided as a further component of the gas scrubber, in which the CO2 can be separated from the solvent. In other words, the CO2 present in the gas is preferably first absorbed by a solvent in an absorber column and then the CO2 is separated from the solvent in a stripping column. If necessary, a pre-cleaning of the gas can also be carried out before the gas is fed into the absorber column, for example by feeding the raw gas into a gas scrubber column provided with packings and scrubbing out side gases, for example H2S and traces of NH3 in the case of biogas. The term “column” is always to be understood in a comprehensive sense here, as well as below, and is expressly intended to include any apparatus with which the desired operation can be carried out, i.e. not only apparatus with packings, but also without packings.

A particularly preferred design is one in which the CO2-laden solvent is fed into the top of the stripping column and trickles downwards there, with a steam feeding device being provided by means of which steam, preferably water steam, can be introduced into the stripping column, as a result of which the CO2 is released and discharged, preferably at the top of the column. It is particularly preferred that the CO2-free solvent can be removed from the stripping column by means of a removal device and returned to the counter-current absorber column, because this ensures that the CO2-free solvent is then removed from the stripping column and returned to the absorber column.

As the above explanations make clear, the preferred integration or coupling of the photobioreactor in a biogas plant and/or in a reator for the gasification of fuels, in particular for the gasification of carbon-containing solid fuels, can thus achieve an advantageous synergistic effect in that the CO2 present or produced there is fed to the photobioreactor in which it is required for carrying out photosynthesis.

The CO2 or the CO2-containing gas can basically be fed continuously, for example in such a way that the CO2 or the CO2-containing gas is continuously introduced, preferably injected, into the selected reactor container(s). Particularly preferred, however, is a set-up in which a pulsing device is provided by means of which the CO2 and/or the CO2-containing gas can be introduced intermittently or pulsatingly into the at least one reactor container, preferably in such a way that individual gas bubbles with a size of less than 3 cm, preferably from 3 μm to 1.0 cm, most preferably from 20 μm to 1.0 cm, can be introduced into the at least one reactor container. The size of these gas bubbles is perfectly sufficient to achieve adequate mixing of the nutrient medium in the respective reactor container with the CO2. As the inventors investigations have shown, the desired ultra-fine distribution of the CO2 can also be very easily controlled, above all, by the pulse frequency and/or the arrangement of the feeding device, in particular by providing a plurality of feed nozzles, and cavitation damage to the reactor container, which can occur in particular with large gas bubbles, can thus also be reliably avoided.

A wide variety of substrates are used in biogas plants, such as liquid manure, corn silage or organic waste. These contain water and organic compounds such as carbohydrates (sugar, starch, hemicellulose, cellulose), proteins, fats and other compounds in varying proportions. A large proportion of these organic compounds is broken down by microorganisms during the anaerobic biogas process. As already mentioned, methane (CH4) and carbon dioxide (CO2) are the most important degradation products in biogas. In addition, small amounts of hydrogen sulfide (H2S) and ammonia (NH3) are initially present. The degradation processes turn the viscous to solid substrate into the liquid to viscous fermentation residue with a high water content, which also contains considerable amounts of nitrogen, phosphorus, potassium, sulfur and trace elements. fermentation residue can thus be regarded as a high-quality organic fertilizer. Accordingly, according to a further particularly preferred embodiment, it is particularly advantageous that a feeding device is provided by means of which fermentation residue can be metered from the biogas plant, in particular from a post-fermenter of a biogas plant, to at least one reactor container, preferably controlled by a control device as a function of a predetermined pH value in the nutrient medium, i.e. as a function of a pH value in the nutrient medium which is optimal for algae growth. Preferably, this pH value is around 7 to 8. The controlled addition of the fermentation residue thus adds a fertilizer to the nutrient medium that significantly promotes algae growth.

According to a particularly preferred concrete embodiment, it is provided that the bottom wall, the partition, the front wall, the rear wall and the overflow wall of at least one reactor container or at least part of the reactor containers, preferably of all reactor containers, extend between two transversely opposite, preferably rectangular and/or plate-shaped side walls and adjoin them, in particular adjoin them in a gas-tight and/or liquid-tight manner and/or are possibly even connected (preferably detachably connected) to them, are even connected to them (are preferably detachably connected). The side walls extend in each case to the top wall and adjoin the latter in order to provide the overall closed structure of the reactor containers. The side walls are adjacent to the top wall in particular in a gas-tight and/or liquid-tight manner. Alternatively or additionally, the side walls may even be connected to the top wall.

A particularly preferred design is one in which the bottom wall of the reactor container is curved in the shape of an arc, with the apex of the curvature being located at the lowest point of the reactor container as seen in the vertical axis direction. Such a curved bottom wall region results in a particularly advantageous geometry that follows the flow pattern and has no dead zones in which material, for example algae material, can accumulate in an undesirable manner. In addition, it is particularly advantageous in this context if the opposite, preferably rectangular and/or plate-shaped side walls extend downwards in the vertical axis direction at least as far as the apex of the bottom wall and form a bottom contact surface. This provides stable reactor containers overall despite the curved bottom wall region.

Particularly preferred in connection with the individual design of the reactor containers is a structure in which each of the reactor containers, which are designed as individual containers, has two separate opposing side walls. As already described above, this forms a separate component that is particularly easy to handle. In principle or as an alternative, however, it is of course also possible for two opposite, large-area side walls to form the side walls for several or all reactor containers. This does not conflict with the individual container concept, which in this embodiment are then formed by the front wall, the partition and the rear wall.

In order to allow light to enter the interior of the reactor containers, it is necessary that, as described above, the front wall or rear wall of the reactor containers associated with lighting elements is/are made translucent at least in this associated area. However, a structure in which at least one or at least a part of the reactor containers and/or the top wall as a whole is/are designed to be translucent, preferably made of a translucent glass or plastic material, is particularly advantageous and simple to manufacture. Further preferred is a structure in which the individual walls are made of the same material and/or in one piece and are thus manufactured cost-effectively.

As already stated, it is also particularly advantageous if the front wall and/or the rear wall and/or the partition and/or the overflow wall region and/or the side walls are rectangular and/or plate-shaped. Such rectangular and/or plate-shaped wall elements can be easily manufactured and permit an overall simple construction of the reactor container, in particular with regard to the formation of common parts.

The single-part or multi-part overflow wall region can, for example, be integrally formed with the front wall and/or the rear wall of a reactor container, in particular with their free end regions. According to a first embodiment, the overflow wall region can, for example, be integrally formed with either the front wall or the rear wall of a reactor container, in particular with their free end regions. To form the common overflow wall region, a free end region of a rear wall or front wall of an immediately adjacent reactor container is then also connected to the overflow wall region. In this embodiment, the overflow wall region then forms an integral part of a single reactor container and the associated wall region of the adjacent reactor container can then simply be connected to the overflow wall region. This achieves a reduction in the variety of components. In addition, such integral composite solutions are easy and inexpensive to manufacture.

An integral design with both the front wall and the rear wall is also possible, for example if the overflow wall region is formed in two parts and has both a front wall side and a rear wall side overflow wall region element that can be connected to each other.

Alternatively, however, the overflow wall region could also be formed in one piece with both the front wall and the rear wall and the interface could be provided elsewhere, i.e. not in the area of the overflow wall region, in the event that separation of individual reactor regions is desired at all.

An integral design in the sense of the two preceding paragraphs preferably means a materially uniform and/or integral connection between the overflow wall region (or its individual elements) and the front and/or rear wall of adjacent reactor containers, so that these reactor containers then form modules which can be easily assembled during final assembly.

It should also be explicitly mentioned at this point that it is not absolutely necessary to assemble the photobioreactor according to the invention from several individual reactor containers, even though this is a preferred embodiment. The photobioreactor according to the invention with its several reactor containers can, of course, also be designed as a single piece as a whole, for example be manufactured as a single piece and as a single material using a 3D printing process. The individual reactor containers then form an overall coherent construct.

According to a further alternative embodiment, the overflow wall region can also be formed by a separate component consisting of one or more parts, which can be firmly connected to the front wall and/or to the rear wall of the two adjacent reactor containers, in particular to theft free end regions. Preferred embodiments are those in which the overflow wall region is formed by a separate component or by several separate components which are connected to the front wall and/or the rear wall of the respective reactor container or reactor containers in the course of a pre-assembly, so that these pre-assembled reactor containers then form pre-assembly modules which are only installed in the course of a temporally downstream final assembly.

According to a particularly preferred embodiment, the overflow wall region is formed by a peripheral frame with a container overflow opening surrounded by the frame. Such a peripheral frame ensures that the overflow wall region is particularly stable. According to a particularly preferred embodiment in this respect, it is provided that a lower frame part region in the vertical axis direction forms a connection region for the free end region of the front wall and/or the rear wall of the respectively assigned reactor containers and/or that an upper frame part region in the vertical axis direction adjoins the top wall, in particular adjoins it in a gas-tight and/or liquid-tight manner and/or is possibly even connected to it (is preferably detachably connected).

The overflow wall region can also have at least one flow guide element projecting into the container overflow opening and/or several container overflow openings, preferably lying next to each other in the transverse direction. The several container overflow openings can have the same or different shapes, According to a particularly preferred embodiment, it is provided that for the formation of several container overflow openings at least one connection web is provided, running between frame parts, preferably at least one connection web running in the vertical axis direction and between frame parts opposite in the vertical axis direction, in particular in a double function as flow guide element. Such an arrangement with at least one flow guide element and/or with several overflow openings and/or with at least one connection web leads to advantageous smaller turbulences and swirls in the area of the overflow wall region, which has a particularly advantageous effect on the flow guidance and distribution of the produced microorganisms in the nutrient medium, since an otherwise possibly existing tendency to settle or accumulate is counteracted.

In connection with such flow guide elements or connection webs, it is of course also possible to form different shapes of container overflow openings, which can also contribute in a targeted manner to forming certain desired flow conditions in the overflow wall region.

The at least one lighting element can be designed in different ways and, for example, have one or more luminaries whose beam angle(s) and thus light cone(s) are either fixed in the mounted state of the at least one lighting element or are also adjustable. In connection with a lighting element whose beam angle and thus light cone is adjustable in the mounted state, the illumination or lighting of the respective reactor chamber of the reactor containers can be adapted and/or changed in an advantageous manner. As the previously made explanations show, the term “lighting element” in the sense of the invention is to be interpreted in an expressly general manner and can be understood to include all suitable lighting means, such as LEDs and/or OLEDs. However, it can just as well be understood to include incandescent lamps, halogen spotlights or fluorescent tubes. The lighting elements used according to the invention, for example LED lights, emit light with an optimal wavelength and intensity that is adapted to the growth of the respective microorganisms, and are also preferably characterized by high energy efficiency.

It is also particularly advantageous if the at least one lighting element is arranged in the gap between the adjacent reactor containers in such a way that differently brightly illuminated areas, in particular as defined light/dark areas, can be formed in the at least one reactor chamber of the adjacent reactor containers illuminated by the at least one lighting element. It is particularly preferred if differently brightly illuminated areas lying one behind the other in the direction of flow are formed, in particular as defined light/dark areas. This is based on the inventor's realization that in the cultivation and propagation of microorganisms, in particular of microalgae, it is of particular advantage not to have permanently uniform illumination as seen in the direction of flow. A permanent uniform illumination can lead to a too high light intensity and thus to a photoinhibition, which results in a reduction of the growth rate of microalgae. Photoinhibition occurs when the intensity of the light exceeds an intensity that ensures a maximum growth rate. Rather, according to the inventor's knowledge, it is particularly advantageous for the growth of microorganisms, especially microalgae, if brighter areas alternate with darker (less brightly illuminated) areas. In other words, the microorganisms or microalgae then find a kind of resting zone in the less brightly illuminated areas, which has an overall positive effect on the growth and reproduction of the microorganisms or microalgae.

Particularly for the formation of such differently brightly illuminated areas, but also in general, an arrangement is advantageous in which several lighting elements are accommodated in the gap between the adjacent reactor containers, spaced apart from one another in the vertical axis direction and/or in the transverse direction. With such an arrangement spaced apart in the direction of the vertical axis and/or in the transverse direction, it is possible to achieve advantageous illumination and lighting of the individual, different areas of the reactor chambers that is precisely matched to the desired individual case. It is particularly preferred that several rows of lighting elements are formed which extend in the transverse direction and are spaced apart from one another in the vertical axis direction, preferably evenly spaced apart from one another.

It is further preferred that the row of lighting elements extending in the transverse direction is formed by several lighting elements spaced apart from one another and/or by light bars.

The spacing of the lighting elements, in particular the row of lighting elements, is between 10 and 40 cm in the vertical axis direction, preferably between 15 and 30 cm, according to a particularly preferred embodiment.

As already explained, in connection with the arrangement of lighting elements according to the invention, a wide variety of lighting conditions can be set and achieved in the individual reactor containers or in their reactor chambers. For example, spaced and successive lighting elements or rows of lighting elements in the direction of the vertical axis can be arranged in such a way that a lighting element/row of lighting elements or a first part of the lighting element/row of lighting elements radiates light through the rear wall (alternatively front wall) of the front (alternatively rear) reactor container, while the next following lighting element/row of lighting elements or the next following part of the lighting elements/row of lighting elements in the direction of the vertical axis emits light through the front wall (alternatively rear wall) of a rear (alternatively front) reactor container. Such an arrangement would of course also be possible alternatively or additionally with respect to the transverse direction.

Alternatively, the lighting elements or row of lighting elements can emit light both through the rear wall of a front reactor container and through the front wall of a rear reactor container. Other group arrangements are also possible in principle.

The explanations just given show that there are a large number of different possibilities for arranging the lighting element(s) in the gap between adjacent reactor containers in order to create lighting conditions tailored to the respective purposes and applications. With the arrangement of the lighting elements in the gap between adjacent reactor containers according to the invention, this is possible in a particularly simple and advantageous manner in contrast to the prior art.

Lighting elements can also be arranged on the top wall, for example on the underside of the top wall. Alternatively or additionally, however, lighting elements can also be arranged on the outer and upper side of the top wall, in conjunction with the preferably used translucent ceiling walls.

Furthermore, a stiffening element, preferably a stiffening element closing the gap downward, can be provided in the gap between the adjacent reactor containers in the transition region from the front and/or rear wall to the bottom wall, which extends over a predetermined length in the transverse direction, in particular extends completely in the transverse direction between opposite side walls and adjoins them. Such an additional stiffening element, which lies at a distance below the overflow wall region, serves to stabilize the structure as a whole.

Furthermore, the partition in the bottom wall side wall area can have a peripheral frame region with a partition-through-flow opening surrounded by the frame region. Preferably, a lower frame part region in the direction of the vertical axis adjoins the bottom wall, in particular adjoins it in a gas-tight and/or liquid-tight manner and/or is connected to it (preferably detachably connected).

The partition also preferably has at least one slow guide element projecting into the partition-through-flow opening and/or a plurality of partition-through-flow openings, preferably adjacent to one another in the transverse direction. In this context, it may be provided, for example, that the plurality of partition-through-flow openings have an identical or different shape. Furthermore, according to a particularly preferred embodiment, it is provided that for the formation of several partition-through-flow openings at least one connection web running between frame parts, preferably at least one connection web running in the vertical axis direction and between frame parts opposite in the vertical axis direction, is provided, in particular in a double function as flow guide element. The same advantages arise here as have already been explained in connection with the container overflow opening of the overflow wall region, i.e., such an arrangement with at least one flow guide element and/or with several through-flow openings and/or with at least one connection web in the region of the partition leads to advantageous smaller turbulences and swirls, which has a particularly advantageous effect on the flow guidance and distribution of the microorganisms produced in the nutrient medium, since an otherwise possibly existing tendency to settle or accumulate is counteracted.

In conjunction with such flow guide elements or connection webs, it is of course also possible to form different shapes of the partition-through-flow openings, which can also contribute in a targeted manner to forming certain desired flow conditions there.

According to a further particularly preferred embodiment of the present invention idea, it is provided that at least one or at least part of the reactor containers has at least one feed nozzle as feeding device, preferably a plurality of feed nozzles spaced apart in the transverse direction as feeding device. A particularly preferred embodiment is one in which at least one or at least some of the reactor containers, preferably all of the reactor containers, have a plurality of feed nozzles spaced apart in the transverse direction, by means of which a medium, in particular CO2 or a medium containing CO2, can be introduced into the reactor container from outside the reactor container. Particularly preferred is an arrangement in which the at least one feed nozzle, preferably a plurality of transversely spaced feed nozzles, is arranged in the bottom wall side region of the reactor container, preferably in the region of the rear reactor chamber on the bottom wall and/or on the rear wall. With such feed nozzles, with which a given medium, in particular CO2 or a CO2-containing medium, can be introduced from outside the reactor container into the reactor container, photosynthesis can be supported in an advantageous manner. By injecting in the bottom wall side area of the reactor container, and here preferably in the area of the rear reactor chamber at the bottom wall or rear wall, it is also advantageously achieved that no material can accumulate there.

Particularly preferably, the at least one feed nozzle is aligned with its mouth opening in the direction of flow, so that the flow of the nutrient medium is supported in the direction of flow when the medium is injected.

The photobioreactor is further preferably designed in such a way that an inlet for the nutrient medium is provided at the front reactor container in the longitudinal direction or flow direction, preferably in the top wall and/or in the front wall and/or in the side wall of the front reactor container in the longitudinal direction or flow direction, which inlet is preferably an inlet by means of which the nutrient medium can be supplied to the front reactor chamber of the front reactor container.

This inlet is preferably coupled with a conveying device, by means of which a part of the nutrient medium, preferably a part of the nutrient medium drawn off from a rear area of the photobioreactor, most preferably a part of the nutrient medium drawn off from the rearmost reactor container in the longitudinal direction or flow direction, can be fed to the front reactor container. The conveying device also serves as a circulation device for the liquid nutrient medium.

In general, the liquid nutrient medium in the photobioreactor is to be circulated by a conveying device of any kind in such a way that a vertical meandering flow is formed through the individual reactor containers.

In principle, the conveying device can also be formed by a conventional pump, but this has the disadvantage that the cell walls of the cultivated microorganisms may be damaged. For this reason, in the solution according to the invention, the conveying device is formed according to a particularly preferred embodiment by an air-lift arrangement, in which a working medium, preferably air, most preferably air enriched with CO2 and/or filtered air, is introduced into a nutrient medium line led to the inlet, so that the working medium conveys the nutrient medium in the direction of the inlet, in particular in the manner of a carrier medium, and conveys it in the direction of the inlet. As the previously made explanations show, the lift arrangement is referred to herein as an “air” lift arrangement, although this does not imply any restriction on the working medium used, which is preferably gaseous. Instead of air as the working medium, another carrier medium, for example an inert gas, can also be used, to give just one further example of a working medium.

Furthermore, according to a particularly preferred embodiment, an outlet for the nutrient medium is provided at the rearmost reactor container in the longitudinal direction or flow direction, preferably in the top wall and/or in the rear wall and/or in the side wall of the rearmost reactor container in the longitudinal direction or flow direction.

The outlet is preferably designed in such a way that it can be used to discharge the nutrient medium from the rear reactor chamber of the rearmost reactor container. The outlet is designed here, for example, as an outlet, in particular as an overflow, and/or is coupled to a removal device by means of which the nutrient medium can be withdrawn from the rearmost reactor container in the longitudinal direction or flow direction, in particular as a function of the density of the microorganisms produced in the photobioreactor, for example in the rearmost reactor container.

The outlet is preferably followed by a continuous belt filter, in particular a self-cleaning continuous belt filter, in which a continuous filter cloth is circulated between a filtering section and a section in which the filtered product is removed from the filter cloth. Once the desired density of the microorganisms produced, in particular the desired algae density, has been reached, the nutrient medium can be at least partially discharged from the photobioreactor via the outlet and the desired product can then be separated from the nutrient medium in a separate station. The continuous belt filter allows particularly gentle recovery of the product, as it does not destroy the cell walls of the recovered microorganisms, unlike the centrifuges normally used for this purpose.

It is understood that the photobioreactor is operated in a closed cycle with respect to the liquid nutrient medium, i.e. the nutrient medium, which is preferably present at the end of the photobioreactor and is provided with microorganisms or microalgae, is fed back to the inlet and this process is repeated until the desired density of the respective product is reached and the renewed (partial) discharge can take place. It goes without saying that due to the consumption of the nutrient medium, new nutrient medium must be added periodically.

According to another particularly preferred embodiment, a heating and/or cooling element is provided on the outside of at least one reactor container, preferably on the bottom wall and/or in the bottom wall side region of the front wall and/or the rear wall and/or the side walls of at least one reactor container, by means of which the nutrient medium accommodated in the reactor container can be tempered.

It is further preferably provided that the top wall, which is formed in one or more parts, is preferably plate-shaped, so that it can be easily handled, for example, in connection with a lifting thereof.

A particularly preferred embodiment is further one in which the top wall is provided with at least one ventilation device, preferably with at least one ventilation fan, by means of which a gas accumulating between the top wall and the nutrient medium, in particular oxygen-containing gas, can be extracted from the interior of the photobioreactor, in particular from the reactor containers, wherein it is optionally provided that a top wall side ventilation device is assigned to each reactor container. This allows in particular to extract the oxygen generated between the top wall and the nutrient medium. In this way, the oxygen partial pressure above the nutrient medium is lowered, which reduces the oxygen content in the nutrient medium. This is advantageous because a too high concentration of oxygen in the nutrient medium would result in a lower productivity of the microalgae biomass, for example. The provision of a ventilation device also has the advantage that condensation on the top wall is minimized, which reduces cleaning and maintenance costs.

Another particularly preferred configuration is one in which, in conjunction with a photobioreactor with multiple reaction vessels, all reactor containers have the same U-shaped basic structure with a front wall and a rear wall of essentially the same height, both of which have a gap distance to the top wall and both of which are surmounted by the partition extending to the top wall and adjoining it. The gap distance to the top wall in the adjacent area of two reactor containers is bridged by the overflow wall region, which extends to and adjoins the top wall. Furthermore, the front wall of the foremost reactor container in the longitudinal direction or flow direction has a first wall- and/or plate-like bridging element which extends to the top wall and adjoins it. The rear wall of the rearmost reactor container in the longitudinal direction or flow direction has a second wall- and/or plate-like bridging element which extends to the top wall and is adjacent thereto. It is further provided that the first and second wall- and/or plate-like bridging element as well as all existing front walls, partitions and rear walls as well as the at least one overflow wall region extend in the transverse direction between the side walls, which likewise extend to the top wall and are adjacent thereto, and are adjacent thereto, so that a closed reactor is formed when the top wall is mounted. With such a structure, the reactor containers are essentially formed as identical parts, so that production and manufacture are substantially simplified.

In the present invention, “adjoining” components to other components (in particular, “adjoining” walls, wall areas or elements to other walls, wall areas or elements) is preferably understood to mean that the components adjoin one another directly and/or adjoin one another without gap distance, i.e. the components are in direct contact with one another when adjoining and, for example, one wall adjoins the other wall in a contact connection and thus adjoins the latter. According to a further particularly preferred embodiment, each of these contact connections can be designed to be gas-tight and/or liquid-tight. Where, in connection with an adjacency of two components as an optional embodiment, reference is also made to a possibly existing connection of the two components, this connection may preferably be designed as a releasable connection, for example as a form-fit and/or snap-in connection, to give just one example. With such a preferred contact and contact connection of the individual components, an overall stable structure results, since the individual walls or wall areas can then extend, for example, up to the top wall and be supported there. This applies equally to the photobioreactor according to the invention as well as to the reactor container according to the invention and the process according to the invention.

The reactor container according to the invention for a photobioreactor, in particular for a photobioreactor as described above, is characterized in that the reactor container is designed as an upwardly open container which, viewed in cross-section, has a U-shape with a front wall extending in the direction of the vertical axis, which is preferably rectangular and/or plate-shaped, and a rear wall which is spaced apart from the front wall in the longitudinal direction and also extends in the direction of the vertical axis, preferably rectangular and/or plate-shaped front wall extending in the direction of the vertical axis and a rear wall which is spaced apart therefrom in the longitudinal direction, likewise extends in the direction of the vertical axis, is preferably rectangular and/or plate-shaped and is connected to one another at the bottom by a bottom wall. In the reactor container there is also provided a partition which is preferably connected to the bottom wall and/or is rectangular and/or plate-shaped and which, starting from the bottom wall, extends upwards in the direction of the vertical axis, so that the partition divides the reactor container, in relation to the longitudinal direction, into a front reactor chamber and a rear reactor chamber. In the partition, in the bottom wall side adjoining and/or connecting region of the partition to the bottom wall, at least one partition-through-flow opening is provided between the front and rear reactor chambers. As has already been explained in connection with the photobioreactor according to the invention, such a reactor container is characterized by a very compact and simple design, whereby this reactor container as a single container can be combined in a simple manner with other reactor containers of the same design or also of a similar design in order to form a photobioreactor with a desired number of reactor containers arranged in cascade one behind the other.

As already explained in connection with the photobioreactor, the reactor container, in particular the free end region of the front wall and/or the rear wall of the reactor container, can be assigned a single-part or multi-part overflow wall region, for example integrally connected thereto or connected thereto as a separate component, the overflow wall region extending in the transverse direction over the reactor container width and having at least one container overflow opening. With regard to the advantages resulting therefrom as well as further design variants in this respect, we refer to the explanations given above.

A further advantage is a design of the reactor container with side walls opposite in the transverse direction, so that the bottom wall, the partition, the front wall, the rear wall and the overflow wall region of the reactor container, which are preferably curved in the form of an arc, extend between the two side walls opposite in the transverse direction and adjoin them, in particular adjoin them there in a gas-tight and/or liquid-tight manner and/or are connected to them. Here again, the side walls are preferably rectangular and/or plate-shaped.

According to a particularly preferred embodiment, the bottom wall of the reactor container is curved in the shape of an arc, the apex of the curvature being located at the lowest point of the reactor container in the direction of the vertical axis. The opposing side walls, which are preferably rectangular and/or plate-shaped, extend downwards in the vertical axis direction at least as far as the apex of the bottom wall to form a ground contact surface. Here, too, we refer to the previously given explanations on the photobioreactor with regard to the further design and the advantages resulting therefrom.

The latter also applies to the further particularly preferred design of the reactor container as being translucent overall, preferably made of a translucent glass or plastic material.

Furthermore, it is advantageous that at least one feed nozzle, preferably a plurality of feed nozzles spaced apart in the transverse direction, is provided on the reactor container, by means of which a medium, in particular CO2 or a medium containing CO2, can be introduced into the reactor container from outside the reactor container. It is preferably provided that the at least one feed nozzle, preferably a plurality of transversely spaced feed nozzles, is arranged in the bottom wall side region of the reactor container, preferably in the region of the rear reactor chamber on the bottom wall and/or on the rear wall. Here, too, we refer to the explanations given previously in connection with the photobioreactor with regard to the further embodiments and/or the advantages resulting therefrom.

Again according to a preferred embodiment, the as such upwardly open reactor container can be closed by at least one, preferably plate-shaped and/or removable, top wall, preferably gas-tight and/or liquid-tight, to form a closed reactor container, in particular in connection with a photobioreactor consisting of several reactor containers.

The reaction vessel also preferably has a U-shaped basic structure with a front wall and a rear wall of essentially the same height, both of which have a gap distance to the top wall and both of which are surmounted by the partition extending up to and adjoining the top wall. The gap distance can be bridged by an overflow wall region and/or by a wall- and/or plate-like bridging element which, in the assembled state, extends to the top wall and adjoins it. It is further provided that the wall- and/or plate-like bridging element and/or the overflow wall region in the assembled state extends in the transverse direction between the side walls, which likewise extend up to and adjoin the top wall, and adjoins the latter, so that a closed reactor container is formed when the top wall is assembled. The advantages resulting from this have also been appreciated in detail previously in connection with the photobioreactor. To avoid further repetition, we therefore refer to the explanations given there.

In addition, the invention claims a biogas plant with at least one photobioreactor according to the invention or a reactor for gasification of fuels, in particular for gasification of carbon-containing solid fuels, with at least one photobioreactor according to the invention. The advantages resulting therefrom have been previously appreciated in detail. In this respect, reference is made to the explanations given there.

And finally, a process according to the invention for the production of microorganisms, in particular of microalgae, by means of a photobioreactor, in particular by means of a photobioreactor as described above, is proposed, in which the photobioreactor is designed as a closed reactor which has a plurality of upwardly open reactor containers which are closed, preferably closed in a gas-tight and/or liquid-tight manner, by a single-part or multi-part, preferably removable, top wall of the photobioreactor, and in which a nutrient medium is accommodated.

According to the invention, at least some of the reactor containers are designed as individual containers which, viewed in cross-section, have a U-shape with a front wall extending in the direction of the vertical axis and a rear wall spaced apart therefrom in the longitudinal direction and also extending in the direction of the vertical axis, which are connected to one another at the bottom by a bottom wall. The reactor containers of the photobioreactor, which are designed as individual containers in this way, are arranged one behind the other, as seen in the longitudinal direction of the photobioreactor (or the direction of flow of the nutrient medium), in such a way that a reactor container at the front, as seen in the longitudinal direction, is connected to a rear wall, which is transparent to light at least in some areas, with the formation of a gap or gap distance to an at least partially transparent rear wall. gap distance adjoins an at least partially light-permeable front wall of a rear reactor container, as seen in the longitudinal direction, the free end regions of the front and rear walls adjoining one another with the formation of the gap having a common overflow wall region which closes the gap from above, with respect to the vertical axis direction, and which has at least one container overflow opening between the adjoining reactor containers. Via this container overflow opening, the nutrient medium can then flow from a front reactor container, as seen in the direction of flow, into a rear reactor container. The front wall and the rear wall of the reactor container(s) are preferably rectangular and/or plate-shaped.

The overflow wall region, which can also be referred to as the overflow wall region element, extends to the top wall and adjoins it. This adjacency is preferably effected in such a way that the overflow wall region adjoins the top wall in a gas-tight and/or liquid-tight manner and/or, if necessary, is even connected to it, preferably detachably connected.

At least one lighting element is accommodated in the gap between adjacent reactor containers (and thus below the overflow wall region as viewed in the vertical axis direction), by means of which light can be emitted through the respectively assigned front and/or rear wall, which is designed to be at least partially translucent, into one of the two adjacent reactor containers or into both adjacent reactor containers.

Furthermore, in each of the reactor containers designed as individual containers, a partition is provided, preferably connected to the bottom wall and/or rectangular and/or plate-shaped, which extends from the bottom wall in the vertical axis direction upwards the top wall and adjoins it, preferably adjoins it in a gas-tight and/or liquid-tight manner and/or is possibly even connected to it (preferably detachably connected), so that the partition divides the reactor container, in relation to the longitudinal direction or flow direction, into a front reactor chamber and a rear reactor chamber. direction of flow, into a front reactor chamber and a rear reactor chamber.

Further, in the partition, in the bottom wall side adjoining and/or connecting region of the partition to the bottom wall, at least one partition-through-flow opening is provided between the front and rear reactor chambers.

With such a structure, a nutrient medium received in the front reactor chamber of a front reactor container can flow through the at least one partition-through-flow opening into the rear reactor chamber of the front reactor container and then continue to flow upwardly from the rear reactor chamber of the front reactor container or through the at least one container overflow opening into a front reactor chamber of a rear reactor container (vertical meandering flow), so that a nutrient medium received in the front reactor chamber of a front reactor container flows through the at least one partition-through-flow opening into the rear reactor chamber of the front reactor container and further flows from the rear reactor chamber of the front reactor container through the at least one container overflow opening into a front reactor chamber of a rear reactor container (vertical meandering flow).

The resulting advantages have already been discussed in detail in connection with the photobioreactor, so that reference is made to the explanations given there in order to avoid repetition.

The invention is explained in more detail below by way of example only, with reference to a drawing.

It shows:

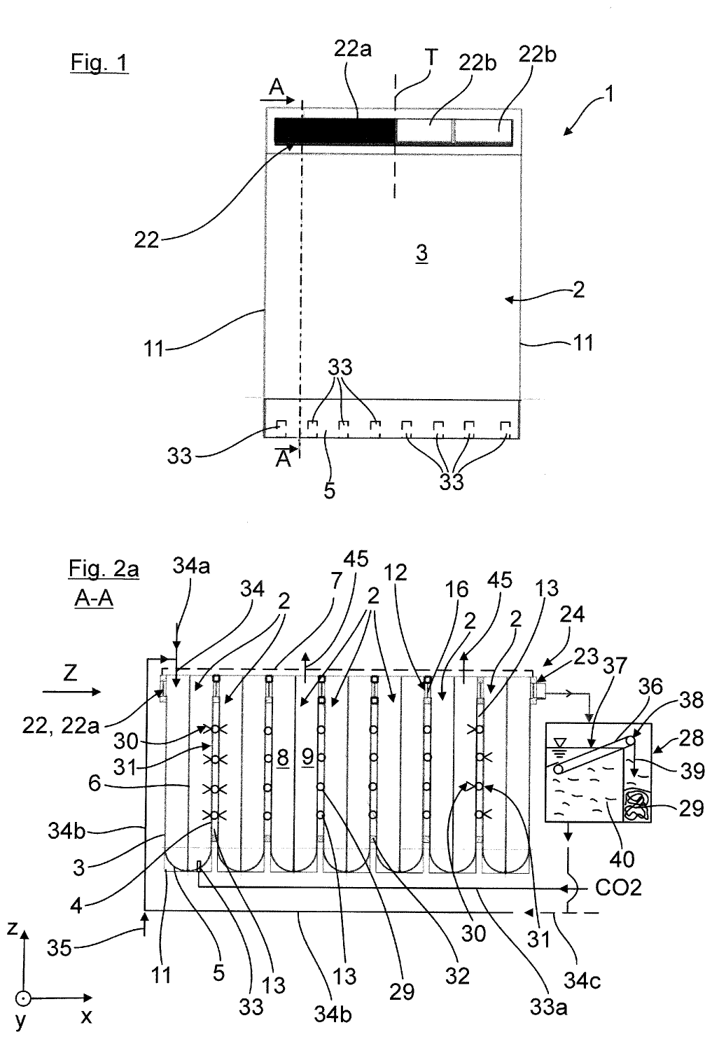

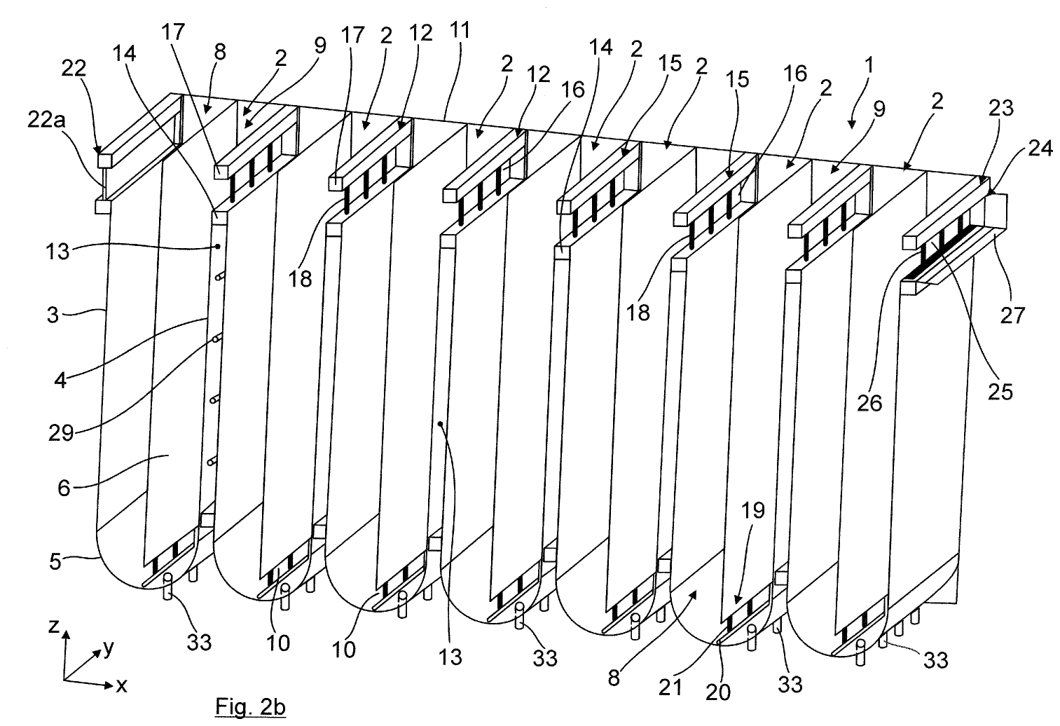

FIG. 1a schematic front view of an exemplary photobioreactor according to the invention, showing a view of the foremost reactor container in the direction of the arrow Z in FIG. 2a,

FIG. 2a schematic longitudinal cross-section along line A-A of FIG. 1,

FIG. 2b schematic perspective sectional view of the photobioreactor of FIG. 2b with features partially omitted,

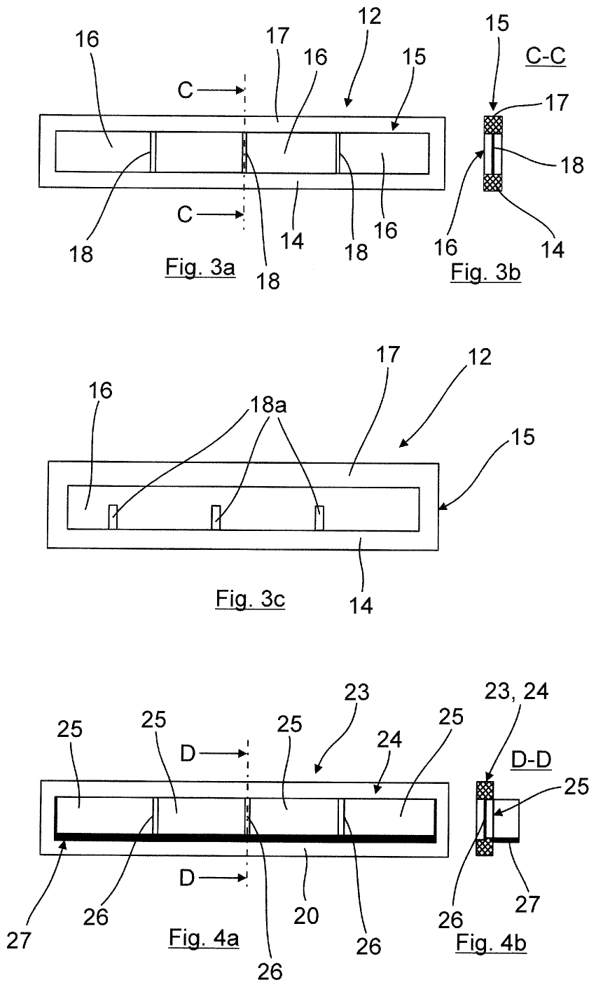

FIG. 3a schematic example of an overflow wall region formed by a separate component,

FIG. 3b schematic sectional view along line C-C of FIG. 3a,

FIG. 3c a schematic representation of another alternative embodiment of the overflow wall region,

FIG. 4a a schematic detail view of a bridging element forming an outlet,

FIG. 4b a section along line D-D of FIG. 4a,

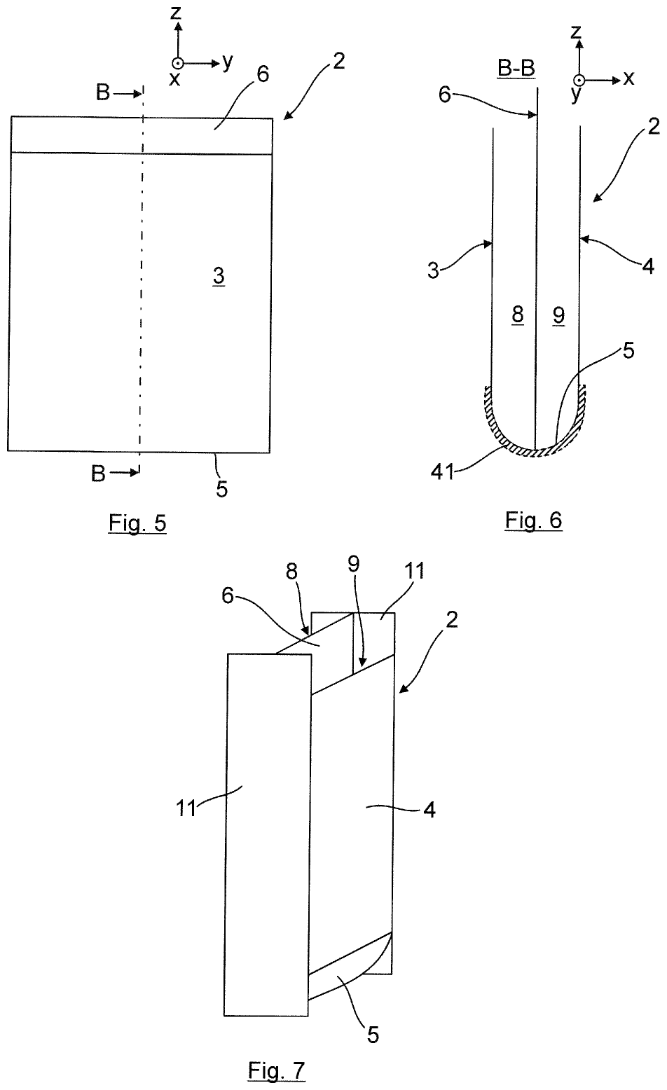

FIG. 5 a schematic front view of a single reactor container,

FIG. 6 a sectional view along line B-B of FIG. 5,

FIG. 7 a perspective view of the single reactor container of FIGS. 5 and 6 with side walls,

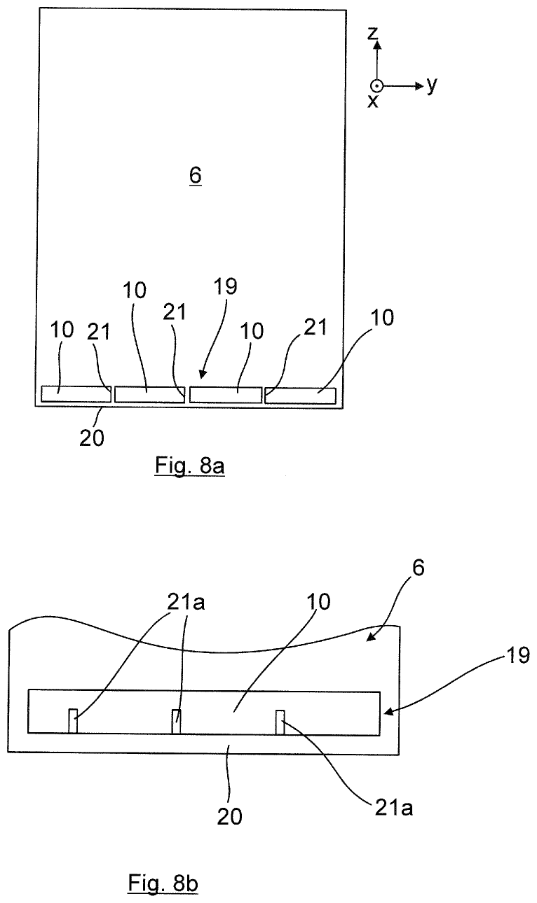

FIG. 8a a magnified detail view of a partition in plan view,

FIG. 8b an alternative embodiment of the partition frame region of FIG. 8a,

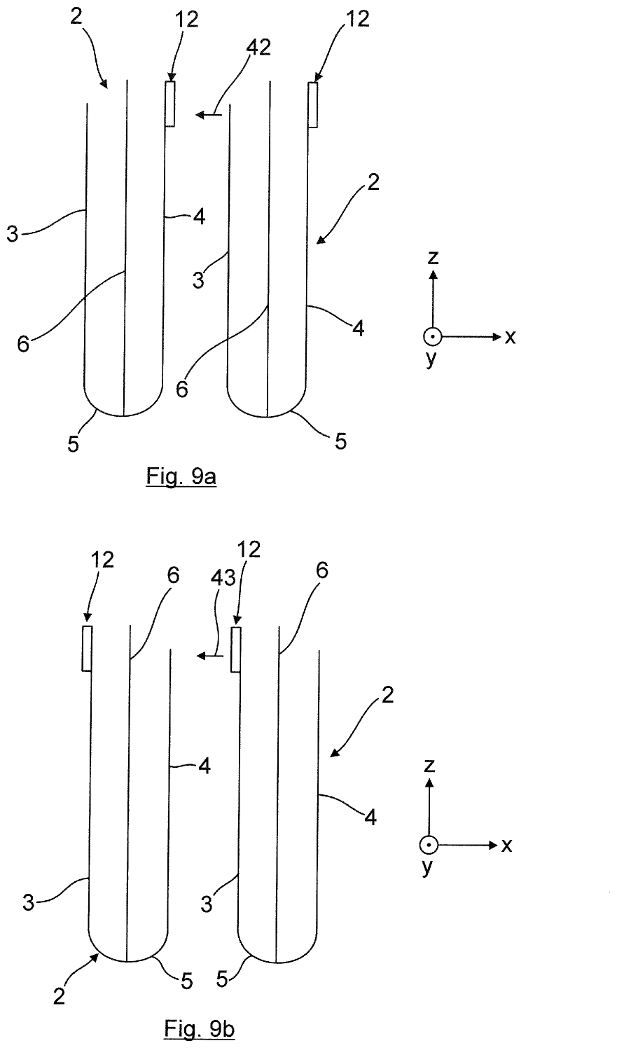

FIG. 9a a schematic representation of an alternative design of an overflow wall region integral with the free end region of the rear wall of a reactor container,

FIG. 9b a schematic representation of an alternative design of an overflow wall region integral with the free end region of a front wall of a reactor container,

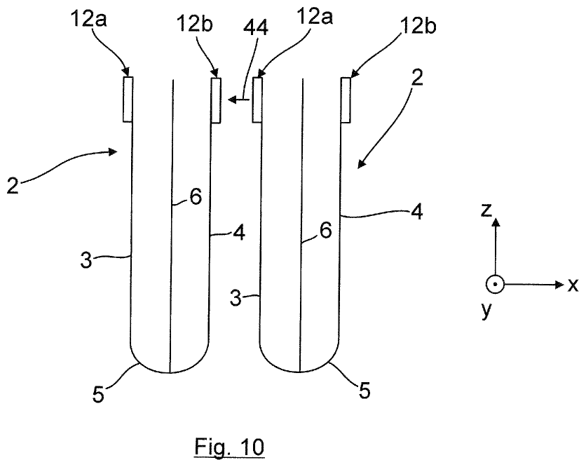

FIG. 10 a schematic representation of a further alternative design of a two-part overflow wall region, the overflow wall region elements of which are integral with the free end region of the rear wall and with the free end region of the front wall of a reactor container,

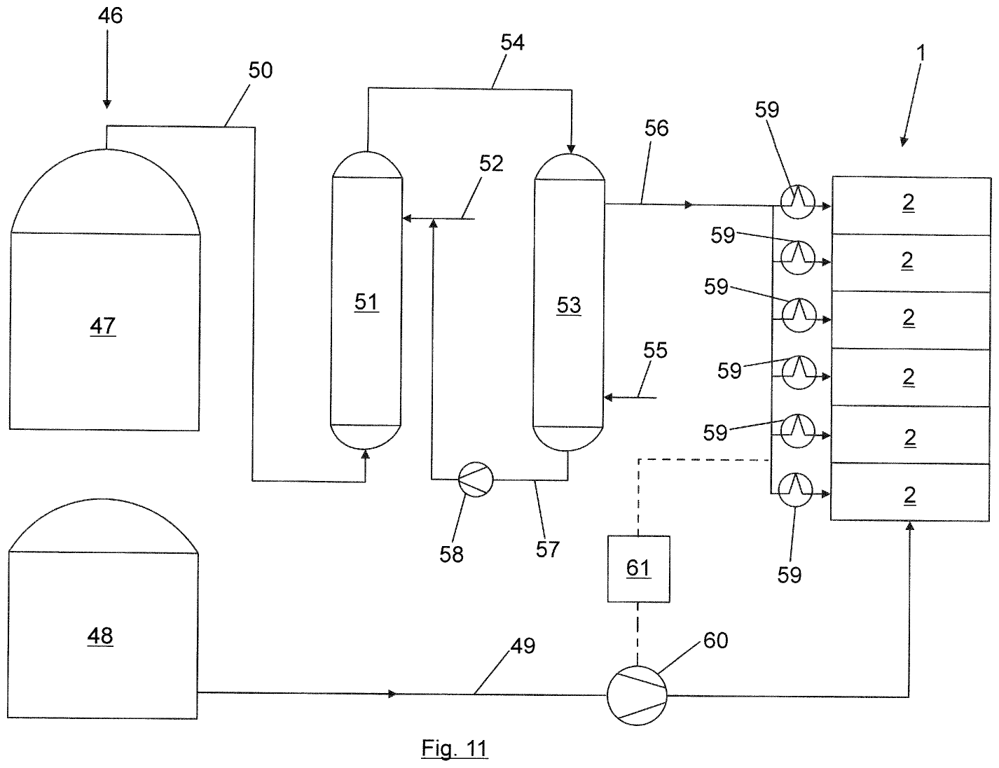

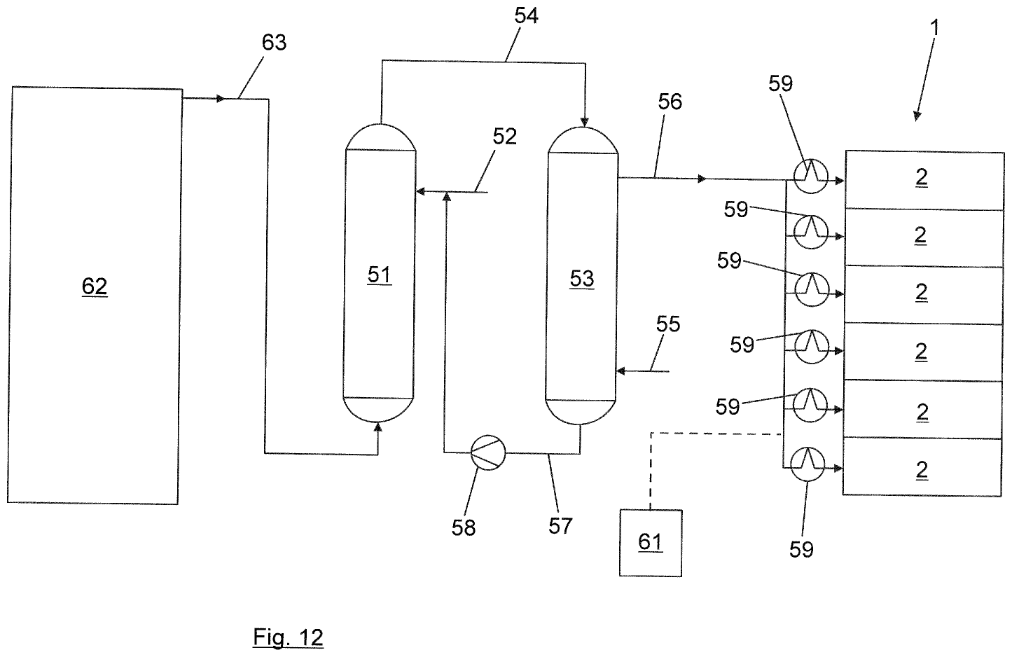

FIG. 11 a schematic representation of a biogas plant according to the invention with a photobioreactor according to the invention, and

FIG. 12 a schematic representation of a reactor according to the invention for gasification of fuels with a photobioreactor according to the invention.

FIGS. 1, 2a and 2b together show an exemplary embodiment of a photobioreactor 1 according to the invention for the production of microorganisms, in particular the production of microalgae. This photobioreactor 1 has, as can be seen in particular from FIGS. 2a and 2b, a plurality of reactor containers 2 in the form of individual containers, in which a nutrient medium is accommodated.

The individual reactor containers 2, as can be seen in particular from the synopsis of FIGS. 5, 6, 7 and 8, all preferably have a substantially identical and/or flow-related U-shaped basic structure, in which the reactor containers 2 are each designed as upwardly open containers and have a front wall 3 extending in the vertical axis direction z and a rear wall 4 spaced apart therefrom in the longitudinal direction x and likewise extending in the vertical axis direction z. The front wall 3 and the rear wall 3 are connected to one another at the bottom by a bottom wall 5. The front wall 3 and the rear wall 3 are connected to each other at the bottom by a bottom wall 5.

Both the front wall 3 and the rear wall 4 are here exemplarily plate-shaped and rectangular, while the bottom wall 5 is here exemplarily curved in the shape of an arc.

The front wall 3 and the rear wall 4 have essentially the same height, as can be seen in particular from FIG. 6, and are surmounted in the vertical axis direction z by a partition 6 arranged here, by way of example, centrally in the reactor container 2. This partition 6 is also exemplarily plate-shaped and rectangular, which can also be seen in particular from FIG. 8a, which shows an individual representation of the partition 6.

In the assembled state (see, for example, FIG. 2a), the partition 6 extends from the bottom wall 5 in the vertical axis direction z upwards to a top wall 7, which is shown here in dashed lines for clarity and is also, for example, plate-shaped and rectangular. The free end region of the partition 6 at the top in the vertical axis direction adjoins the top wall 7, preferably in a gas-tight and/or liquid-tight manner. If necessary, the partition 6 can also be connected to the top wall 7, in particular detachably. The top wall 7 is shown here in one piece, but can also be made in several pieces if necessary.

As can be seen in particular from FIGS. 2a, 2b and 6, the partition 6 divides the reactor container, with reference to the longitudinal direction x, into a front reactor chamber 8 and a rear reactor chamber 9.

In the partition 6, as can be seen in particular from FIG. 8a, in the bottom wall side adjoining and/or connecting region of the partition to the bottom wall 5, several partition-through-flow openings 10 are formed, which allow the nutrient medium to flow over from the front reactor chamber 8 into the rear reactor chamber 9.

The partition 6, like the front wall 3 and the rear wall 4, extends in the transverse direction y between two side walls 11, which are opposite in the transverse direction y and are likewise merely rectangular and plate-shaped by way of example here, and which, as can be seen in particular from FIGS. 2a, 2b and 7, extend in each case as far as the top wall 7 and adjoin the latter, in particular adjoin it in a gas-tight and/or liquid-tight manner or are possibly even connected to the latter. The latter also applies, of course, to the adjacency of the front wall 3, the partition 6 and the rear wall 4 to the side walls 11.

It should be noted at this point that the top wall 7 is preferably designed as a removable top wall, so that either no connection may be provided or a detachable connection must be provided between the top wall 7 and the walls or wall areas adjacent to it.

As can be seen in particular from FIGS. 2a and 7, the apex of the curvature of the bottom wall 5 of the reactor container is located at the lowest point of the reactor container 2 as seen in the vertical axis direction z, so that the opposite side walls 11 extend downward as seen in the vertical axis direction z at least to the apex of this bottom wall 5 and thus form a ground contact surface.

In the embodiment shown in FIGS. 2a and 7, each individual reactor container 2 has two separate opposite side walls 11. In FIG. 2b, however, an alternative variant is shown in which two opposite large-area side walls 11 each form the side wads for several or, in the case of FIG. 2b, for all reactor containers 2.

Both the individual reactor containers 2 and the top wall 7 are preferably designed to is be translucent as a whole, for example made of a translucent glass or plastic material.

As can be seen from FIG. 6 in conjunction with FIGS. 2a and 2b, all reactor containers 2 have the same basic U-shaped structure with a front wall 3 and rear wall 4 of the same height, both of which have a gap distance to the top wall 7 and both of which are surmounted by the partition 6 extending to the top wall 7.

In order to bridge the gap distance to the top wall 7, the photobioreactor 1 has an overflow wall region 12 in the adjoining area of two reactor containers 2, which is described below and which, viewed in the vertical axis direction z, extends as far as the top wall 7 and in the transverse direction y between the opposing side walls 11 and adjoins them in each case, in particular adjoins them in a gas-tight and/or liquid-tight manner and/or is possibly even connected to them.

This overflow wall region 12 is formed in the present case merely by way of example by a separate component (see FIG. 3a), which is firmly connected to the front wall 3 and the rear wall 4 of two adjacent reactor containers 2 (see FIGS. 2a and 2b). As can be seen from FIGS. 2a and 2b, the individual reactor containers 2 are arranged one behind the other in the longitudinal direction x in such a way that a front reactor container 2, as seen in the longitudinal direction x, with a light-permeable rear wall adjoins a light-permeable front wall 3 of a rear reactor container 2, as seen in the longitudinal direction x, forming a gap 13 as an assembly clearance. In the example shown here, the free end regions of the front and rear walls 3, 4 assigned to the overflow wall region 12 are each connected to a lower frame part region 14 of a peripheral frame 15 of the overflow wall region 12, in particular connected in a gas-tight and/or liquid-tight manner. As a result, the mutually associated front and rear walls 3, 4 of the adjacent reactor containers 2 each have a common overflow wall region 12, which closes the gap 13 from above in relation to the vertical axis direction z and here only has several container overflow openings 16 as an example.

As can be seen in particular from FIG. 3a, the lower frame part region 14 in the vertical axis direction z forms the connection region for the free end regions of the front walls 3 and rear walls 4 of the associated reactor containers 2, while an upper frame part region 17 in the vertical axis direction z adjoins the top wall 7, in particular adjoins it in a gas-tight and/or liquid-tight manner and/or is possibly even connected to it, preferably detachably connected.

The plurality of container overflow openings 16 adjacent to each other in the transverse direction are formed here by a plurality of connection webs 18 extending in the vertical axis direction z between the upper frame part region 17 and the lower frame part region 14, which preferably simultaneously form flow guide elements.

Alternatively, however, only a single container overflow opening 16 without flow guide elements or connection webs 18 could be provided (not shown), or a container overflow opening 16 could be provided into which one or more flow guide elements 18a protrude, as shown only by way of example in FIG. 3c.

As can be seen in particular from the synopsis of FIGS. 2a and 2b, this arrangement of the overflow wall region 12 between the associated front and rear walls 3, 4 of adjacent reactor containers 2 results in an upper overflow region, relative to the vertical axis direction z, through which a nutrient medium can flow or overflow from a rear reactor chamber 9 of a front reactor container 2 into a front reactor chamber 8 of a rear reactor container 2.

According to an alternative embodiment, however, the overflow wall region 12 can also be integrally formed with the free end region of the rear wall 4 of the reactor container 2. This is shown schematically in FIG. 9a. In this case, a free end region of a front wall 3 of a directly adjacent reactor container 2 is also connected to the overflow wall region 12 to form the common overflow wall region 12 (see arrow 42).

According to a further alternative embodiment, however, the overflow wall region 12 can also be integrally formed with the free end region of the front wall 3 of the reactor container 2. This is shown schematically in FIG. 9b. In this case, a free end region of a rear wall 3 of a directly adjacent reactor container 2 is also connected to the overflow wall region 12 to form the common overflow wall region 12 (see arrow 42).

It is also evident that an embodiment according to FIGS. 9a and 9b again results in identical parts, since the reactor containers 2 only have to be rotated by 180° in order to form an overflow wall region 12 arranged on a front wall 3 or on a rear wall 4 in each case.

The latter also applies to the further alternative embodiment shown in FIG. 10, in is which the overflow wall region 12 is formed in multiple parts and a first front wall side overflow wall region element 12a is integrally formed with the free end portion of the front wall 3 and a second rear wall side overflow wall region element 12b is integrally formed with the free end portion of the rear wall 4 of the reactor container 2. The front wall side overflow wall region element 12a and the rear wall side overflow wall region element 12b of two adjacent reactor containers 2 are then joined together to form the common overflow wall region 12, as indicated by the arrow 44 in the FIG. 10 illustration. In principle, such a solution would also be possible with overflow-wall region elements 12a, 12b, which are designed as separate components and must first be connected to the free end regions of the associated walls as part of a pre-assembly process.

A similar structure to the overflow wall region 12 is also shown by partition 6 in conjunction with its partition-through-flow openings 10, which have already been briefly discussed earlier.

As can be seen in particular from FIG. 8a, the partition 6 has a peripheral frame region 19 in the bottom wall side wall area, the lower frame part region 20 of which in the vertical axis direction 7 adjoins the bottom wall 6, in particular adjoins it in a gas-tight and/or liquid-tight manner and/or is even connected to it if necessary, preferably detachably connected.

Here, too, the partition 6 has, by way of example, a plurality of partition-through-flow openings 10 adjacent to one another in the transverse direction y, which are formed by a plurality of connection webs 21 extending between opposing frame parts, which preferably simultaneously form flow guide elements.

Thus, the nutrient medium can also flow from the front reactor chamber 8 into the rear reactor chamber 9, resulting in an overall vertical meandering flow pattern of the nutrient medium in photobioreactor 1.

Alternatively, however, only a single partition-through-flow opening 10 without flow guide elements or connection webs 21 could be provided (not shown), or a partition-through-flow opening 10 could be provided into which one or more flow guide elements 21a protrude, as shown merely by way of example in FIG. 8b.

As can be seen in particular from FIGS. 2a and 2b, the front wall 3 of the foremost reactor container 2 in the longitudinal direction x or flow direction has a first wall- and/or plate-like bridging element 22 which extends from the free end region of the front wall 3 to the top wall and adjoins the latter, in particular adjoins it in a gas-tight and/or liquid-tight manner and/or is possibly even connected to the latter.

The same applies in an analogous manner to the rear wall 4 of the rearmost reactor container 2 in the longitudinal direction x or flow direction, which has a second wall- and/or plate-like bridging element 23 that also extends to the top wall 7 and adjoins it, in particular adjoins it in a gas-tight and/or liquid-tight manner and/or is possibly even connected to it.

With such a structure of a photobioreactor 1, in which bridging elements 22, 23 are used in addition to overflow wall regions 12 in the adjacent area of two reactor containers 2 at the opposite free end sides of the photobioreactor 1, it is ensured that reactor containers with the same basic structure can be used in each case, regardless of the respective position of the reactor containers in the photobioreactor.

The first and second bridging elements 22 and 23 are preferably separate components that must be connected to the respective wall area of the reactor container 2. However, this is not a mandatory measure. In principle, it would also be possible to design the front wall of the foremost reactor container 2 and the rear wall of the rearmost reactor container 2 from the outset with a height such that the front wall 3 of the foremost reactor container 2 and the rear wall 4 of the rearmost reactor container 2 extend upward in the vertical axis direction z to the top wall 7 and adjoin it there.

As can be further seen in particular from the synopsis of FIGS. 4a and 4b, the second bridging element 23 may be formed substantially analogously to the overflow wall region 12 of FIGS. 3a and 3b to form, for example, an outlet 24 having at least one outlet opening 25, preferably a plurality of outlet openings 25. Again, the plurality of outlet openings 25 are formed by providing connection webs 26 between opposing frame part regions. In addition, a nozzle-like overflow connection 27 extends outwardly from the mouth region of the outlet 24, so that a defined overflow is created, for example to an adjoining further photobioreactor of essentially identical or the same design, or also as an outlet to a continuous belt filter 28 shown here as an example. This continuous belt filter 28 will be described in more detail below.

The fact that the partition 6 and the overflow wall region 12 and possibly also the bridging elements 22, 23 each extend to the top wall 7 and adjoin it, preferably in a contact and abutment connection without gap distance, preferably adjoining it in a gas-s tight and/or liquid-tight manner, results in an overall stable structure, since the individual walls or wall regions then extend to the top wall 7 and can be supported there, for example can also be accommodated in a groove-shaped recess, for example can also be detachably latched. In addition to a particularly advantageous seal, the latter also permits a functionally reliable arrangement of the top wall 7 or the individual walls and wall areas in the respective desired position. For the partition 6, this also applies in an analogous manner to its connection to the bottom wall 5.

As can be seen in particular from FIG. 2a in conjunction with FIG. 2b, a plurality of lighting elements 29 are arranged in each case in the gap 13 between the adjacent reactor containers 2, exemplified here in such a way that a plurality of rows of lighting elements 29a, 29b, 29c and 29d are provided which extend in the transverse direction y and are spaced apart from one another in the vertical axis direction z, preferably uniformly spaced apart from one another, as exemplified here.

The individual rows of lighting elements 29a, 29b, 29c and 29d can, for example, be lighting elements 29 in the form of LED light bars, to name just one example, whose LEDs can emit light both through the front wall 3 and through the rear wall 4 of two adjacent reactor containers into the respective reactor chambers of the reactor containers 2. This is shown only by way of example in connection with the two reactor containers 2 on the left in the image plane of FIG. 2a.

Alternatively, the lighting elements 29, for example as LED light bars, can also be arranged or designed in the gap 13 in such a way that light, as shown in connection with the two reactor containers 2 on the right in the image plane of FIG. 2a, is emitted alternately only into one of the two associated reactor containers 2. In the example shown on the right-hand side of FIG. 2a, which is not to be understood as conclusive, the lighting elements 29 arranged one above the other in the direction of the vertical axis z only emit light alternately (seen from top to bottom) through the rear wall 4 of the front reactor container 2, then through the front wall 3 of the rearmost reactor container 2, then again through the rear wall 4 of the front reactor container 2 and finally again through the front wall 3 of the rearmost reactor container 2. It goes without saying that other arrangements and illuminations are also possible at any time.

These two lighting situations shown in FIG. 2a merely by way of example using the lighting elements 29 and the row of lighting elements 29a to 29d are intended to show that it is particularly advantageous to form differently brightly illuminated areas 30, 31 in the respective reactor chambers 8, 9, these differently brightly illuminated areas 30, 31 preferably being areas lying one behind the other in the flow direction of the vertical meander flow. In the present example case, areas 30 are thus more brightly illuminated than areas 31, resulting in a certain light-dark effect which has a beneficial effect on the growth of microorganisms, in particular on the growth of phytoplankton such as microalgae.

In the solution according to the invention, the overflow through the partition 6 between the individual reactor chambers 8, 9 or the overflow through the overflow wall region 12 between the individual reactor containers 2 then takes place in an advantageous manner through overflow openings 10, 16 which are adapted to the respective application and which can be geometrically designed in such a way that a targeted influencing of the flow conditions of the vertically meandering flow can be achieved in the respective overflow region, for example in such a way that targeted slight turbulence or swirling is caused there, which counteracts, for example, a settling movement of generated microorganisms without impairing the flow course as such. turbulences can be caused, which counteracts, for example, a settling movement of generated microorganisms without impairing the flow course as such.

As can be seen from FIGS. 2a and 2b, a stiffening element 32 can be provided in the gap 13 between the respective adjacent reactor containers, preferably in the area above the transition region from the front and/or rear wall 3, 4 to the bottom wall 5, for example a stiffening element 32 closing the gap 13 downward. This stiffening element 32 can extend over a predetermined length in the transverse direction y, for example also extending completely between the opposite side walls 11.

As can also be seen from the synopsis of FIGS. 1, 2a and 2b, a plurality of transversely spaced feed nozzles 33 are provided in the bottom wall side area of the reactor containers 2, in this case in the area of the rear reactor chamber 9 on the bottom wall 5, by means of which a medium, in particular CO2 or a medium containing CO2, can be introduced into the reactor container 2 from outside the reactor container 2.

The feed nozzles are preferably aligned with their mouth opening in the direction of flow (compare in particular FIG. 2a), so that the flow of the nutrient medium is supported in the direction of flow when the medium is injected. In addition, such injection can also reliably prevent deposits in the rear reactor chamber, in particular in the bottom wall region.

As can be seen in particular from the synopsis of FIGS. 1 and 2a, the first bridging element 22 can be designed differently, for example as a closed wall element 22a (to the left of the dividing line T) or, analogously to the overflow wall region 12, can be provided with overflow openings 22b (to the right of the dividing line T). This depends, for example, on how the photobioreactor 1 is specifically used or employed. If the photobioreactor 1 is used as a single reactor or as the first reactor of a reactor cascade, then the first bridging element 22 can be designed as a closed wall element 22a and the nutrient medium is then fed in via the inlet 34, which is only shown schematically in FIG. 2a.

On the other hand, in case the photobioreactor 1 is part of a reactor cascade and does not form the first photobioreactor here, it may be provided that the first bridging element 22 is provided with the overflow openings 22b, which are then fluidically coupled to the outlet 24 of a preceding photobioreactor 1, preferably via the overflow connection 27 to which the first bridging element 22 is coupled (not shown in detail here).

In connection with FIGS. 2a and 2b, the first bridging element 22 is here exemplarily formed as a closed wall element 22a.

As can be further seen from FIG. 2a, the inlet 34 can further be coupled to a feed line 34a by means of which fresh nutrient medium 34a can be fed to the photobioreactor 1 at given times.

The inlet 34 is also connected to a nutrient medium line designed here as return line 34b, which in this example leads from the last reactor container 2 and by means of which the nutrient medium is circulated via the inlet 34. In principle, a pump can be connected to the return line 34b as a conveying device. Particularly preferably, is however, the conveying device in the solution according to the invention is formed by an air-lift arrangement 35, in which a specific working medium, preferably air, most preferably air enriched with CO2 and/or filtered air, is introduced into the return line 34b led to the inlet 34, which conveys the nutrient medium in the direction of the inlet 34.

As shown further, in this circulation of the nutrient medium, part of the nutrient medium is preferably drawn off from the rear-most reactor container 2 in the longitudinal direction x or flow direction and then fed back to the front-most reactor container 2 in the longitudinal direction x b or flow direction. However, this can also be deviated from if necessary, for example in such a way that several return lines are provided which branch off from several reactor containers and are led to the inlet. Likewise, an inlet can be provided alternatively or additionally in connection with other or further reactor containers.

The air-lift arrangement 35 thus serves here simultaneously as a circulation device for the liquid nutrient medium in the photobioreactor 1, i.e. as a circulation device for guiding the nutrient medium vertically meandering through the photobioreactor 1 in the desired manner. As has been pointed out before, such an air-lift arrangement 35 is particularly gentle on the product. However, the invention can in principle be implemented with any type of circulation device.

In the schematic, principal embodiment shown in FIG. 2a, the photobioreactor 1 is followed by the continuous belt filter 28, in which an endless filter cloth 36 is circulated between a filtering section 37 and a section 38 in which the filtered product 39 is removed from the filter cloth 36. This is shown only very schematically in FIG. 2a.

As can also be seen from FIG. 2a, the filtered nutrient medium 40 can be returned to the nutrient medium circuit via a further return line 34c, if necessary.

FIG. 2a further shows that the feed nozzles 33 can also be coupled to a feed line 33a, via which, for example, CO2-enriched medium, for example CO2-enriched air, can be supplied.

It is understood that valves, backflow preventers and other blocking elements or control elements can of course be arranged in the respective media-carrying lines in the usual manner, with which control or regulation of the media flow takes place.

Furthermore, a heating and/or cooling element 41 can be arranged on the bottom wall of each of the reactor containers, by means of which the nutrient medium contained in the respective reactor container 2 can be appropriately tempered. This is shown only by way of example and schematically in FIG. 6.

And finally, the top wall 7 may be provided with one or more ventilation device(s) 45 formed, for example, by ventilation fans. This is only shown extremely schematically and by way of example in FIG. 2a. By means of these ventilation devices 45, gas accumulating between the top wall 7 and the nutrient medium, in particular oxygen-containing gas, can be extracted from the interior of the photobioreactor 1, in particular from the reactor containers 2. In principle, a top wall side ventilation device 45 can be assigned to each reactor container 2.

FIG. 11 schematically shows an exemplary representation of a biogas plant 46 with a photobioreactor 1 according to the invention, which, as described above, has a plurality of reactor containers 2. The biogas plant 46 is shown here only extremely schematically and has, among other things, a fermenter 47, in which the actual biogas is produced, and a final storage 48, in which a fermentation residue 49 is located.

As shown in FIG. 11, the biogas 50 is withdrawn from the digester 47 of the biogas plant 46 and fed to a counter-current absorber column 51, in which the CO2 present in the gas is absorbed by a circulating solvent 52. Further, a stripping column 53 is provided in which the CO2 is separated from the solvent. Specifically, the CO2-laden solvent 54 is fed into the head of the stripping column 53, where it trickles down. A steam feeding device 55 is also provided, by means of which steam, preferably water steam, can be introduced into the stripping column 53, whereby the CO2 or, depending on the degree of purification, a CO2-containing gas 56 is released and discharged, preferably at the column head.

The CO2-free solvent 57 is then withdrawn from the stripping column 53 by a removal device 58 and returned to the counter-current absorber column 51.

Furthermore, a plurality of pulsing devices 59 associated with each individual reactor container 2 is provided by way of example, by means of which the CO2 or the CO2-containing gas 56 can be introduced intermittently or pulsatingly into the respective reactor container 2, in principle at any desired point, but preferably on the bottom side, in order to generate individual gas bubbles of a defined size, for example from 3 μm to 1.0 cm.

Furthermore, a feeding device 60 is provided by means of which fermentation residue 49 can be fed to one or more reactor container(s) 2 as fertilizer for the nutrient medium and/or for setting a desired pH value in the nutrient medium. The supply is preferably controlled by a control device 61 shown here only by way of example, for example as a function of a predetermined pH value in the nutrient medium. The control device 61 can also be used, as shown here only schematically, to control the CO2 supply by means of the pulsing devices 59.

Finally, FIG. 12 shows a set-up similar to FIG. 11, but with the difference that instead of a biogas plant 46, a reactor 62 for gasification of fuels, e.g. coal or biomass (e.g. wood as biomass), is used here and accordingly no feeding of the photobioreactor 1 with a fermentation residue takes place. Instead of the CO2-containing biogas, CO2-containing synthesis gas is fed to the counter-current absorber column 51.

It is understood that CO2-containing biogas from a biogas plant and CO2-containing so synthesis gas from a reactor for gasification of fuels can of course also be fed to the photobioreactor 1, although this is no longer explicitly shown. Both CO2-containing gas streams can be fed to different or common gas scrubbers as described above.

### LIST OF REFERENCE SINS

1 photobioreactor
2 reactor container
front wall
4 rear wall
5 bottom wall
6 partition
7 top wall
8 front reactor chamber
9 rear reactor chamber
10 partition-through-flow openings
11 side walls
12 overflow wall region
12a first overflow wall region element
12b second overflow wall region element
13 gap
14 lower frame part region
15 frame
16 container overflow openings
17 upper frame part region
18 connection webs
18a flow guide element
19 frame region
20 lower frame part region
21 connection webs
21a flow guide element
22 first bridging element
22a closed wall element
22b overflow openingen
23 second bridging element
24 outlet
25 outlet openings
26 connection web
27 overflow connection
28 continuous belt filter
29 lighting elements
29a row of lighting elements
29b row of lighting elements
29c row of lighting elements
29d row of lighting elements
30 more brightly illuminated region
31 more darkly illuminated region
32 stiffening element
33 feed nozzles
33a feed line
34 inlet
34a feed line
34b return line
34c return line
35 air-lift arrangement
36 filter cloth
37 filtering section
38 section
39 filtered product
40 filtered nutrient medium
41 heating and/or cooling element
42 arrow
43 arrow
44 arrow
45 ventilation device
46 biogas plant
47 fermenter
48 final storage
49 fermentation residue
50 biogas
51 counter-current absorber column
52 solvent
53 stripping column
54 CO2 laden solvent
55 steam
56 CO2 resp. CO2-containing gas
57 CO2-free solvent
58 removal device
59 pulsing device
60 feeding device
61 control device
62 reactor
63 synthesis gas

1. Photobioreactor for the production of microorganisms,
wherein said photobioreactor is designed as a closed reactor comprising:
a plurality of upwardly open reactor containers which are closed by a single-part or multi-part, top wall of the photobioreactor and in which a nutrient medium can be accommodated,
wherein at least a part of the reactor containers is an individual container which has a U-shaped cross section with a front wall extending in the direction of a vertical axis and a rear wall spaced apart therefrom in a longitudinal direction and likewise extending in the direction of the vertical axis, which rear walls are connected to one another at a bottom by a bottom wall so that the reactor containers of the photobioreactor, are individual containers arranged one behind the other in the longitudinal direction so that a reactor container at the front adjoins a front wall of a reactor container at the rear forming a gap therebetween;
wherein the free end regions of the front and rear walls adjoining one another with the formation of the gap have a common overflow wall region which closes the gap from above with respect to the vertical axis direction and which has at least one container overflow opening between the adjoining reactor containers;
that the overflow wall region extends up to and adjoins the at least one top wall,
wherein at least one lighting element is positioned in the gap between adjacent reactor containers, by means of which lighting element light can be emitted through the respectively associated front and/or rear wall, which is of light-permeable design at least in regions, into one of the two adjacent reactor containers or into both adjacent reactor containers;
wherein a partition is provided in each of the individual reactor containers so that said partition extends from the bottom wall upwardly in the vertical axis direction to adjoin the top wall, so that the partition divides the reactor container in the longitudinal direction, into a front reactor chamber and a rear reactor chamber;
wherein the bottom wall side adjoining the region of the partition to the bottom wall, so that at least one partition-through-flow opening is provided between the front and the rear reactor chamber;
wherein at least one or at least a part of the reactor container has at least one feeding device by means of which a CO2-containing medium can be introduced from outside the reactor container into at east one reactor container, and
wherein the CO2-containing medium is selected from the group consisting of a CO2-containing gas or a CO2 obtained from a CO2-containing gas.

2. Photobioreactor according to claim 1, wherein the photobioreactor is a component of a reactor for gasification of carbon-containing solid fuels, and wherein the CO2-containing gas is a CO2-containing synthesis gas from a reactor for gasification of carbon-containing solid fuels.

3. Photobioreactor according to claim 1, wherein the photobioreactor is part of a biogas plant and wherein the CO2-containing gas is a CO2-containing biogas from a biogas plant.

4. Photobioreactor according to claim 2, wherein a gas scrubber is provided, by means of which the CO2-containing gas can be separated from the gas by the process selected from the group consisting of gas scrubbing and chemical absorption.

5. Photobioreactor according to claim 4, wherein the gas scrubber comprises an absorber column, in which the CO2 present in the gas is absorbed by a solvent, and
wherein the gas scrubber further comprises a stripping column in which the CO2 can be separated from the solvent.

6. Photobioreactor according to claim 5, wherein the solvent charged with CO2 is guided into the head of the stripping column and trickles downwardly, and
wherein a steam feeding device is provided, by means of which steam can be introduced into the stripping column, whereby the CO2 is released and discharged at the column head, wherein it is optionally provided that the CO2 free solvent can be withdrawn from the stripping column by means of a removal device and returned to the absorber column.

7. Photobioreactor according to any one of the preceding claims, wherein a pulsing device is provided by means of which the CO2 and the CO2-containing gas can be introduced intermittently or pulsating y into the at least one reactor container in such a way that individual gas bubbles with a size of less than 3 cm can be introduced into the at least one reactor container.

8. Photobioreactor according to one of claim 3, wherein a feeding device is provided, by means of which at least one reactor container can be fed fermentation residue from the biogas plant, from a post-fermenter and a final storage of the biogas plant, controlled by a control device as a function of a predetermined pH value in the nutrient medium.

9. Photobioreactor according to claim 1, wherein, the bottom wall, the partition, the front wall, the rear wall and the overflow wall region of the reactor container, herein the bottom wall is curved in the form of an arc, extending between two opposing side walls in the transverse direction and adjoin the opposing side walls, and
wherein the side walls each extend up to and adjoin the top wall.

10. Photobioreactor according to claim 9, wherein the bottom wall of the reactor container is curved in the shape of an arc, the apex of the curvature being located at the lowest point of the reactor container in the direction of the vertical axis, and
wherein the opposite side walls extend downwardly in the vertical axis direction at least as far as the apex of the bottom wall and form a bottom contact surface.

11. Photobioreactor according to claim 9, wherein each
individual reactor container has two separate opposite side walls.

12. Photobioreactor according to any one of the preceding claims, wherein the reactor container and the top wall are translucent as a whole.

13. Photobioreactor according to claim 1, wherein the front wall and the rear wall and the partition and the overflow wall region and the side walls have a shape selected from the group consisting of rectangular and plate-shaped.

14. Photobioreactor according to claim 1, wherein the overflow wall region formed in one or more parts is integrally for med with the free end region of the front wall and the free end region of the rear wall of the reactor container.

15. Photobioreactor according to claim 14, wherein the overflow wall region is integrally formed with elements selected from the group consisting of the free end portion of the front wall and the free end portion of the rear wall of the reactor container; and
wherein elements selected from the group consisting of a free end portion of rear wall, and a free end portion of a front wall of an immediately adjacent reactor container is also connected to the overflow wall region for forming the common overflow wall region.

16. Photobioreactor according to claim 1, wherein the overflow wall region formed in one or more parts is formed by a separate component which can be connected to the front wall and the rear wall of the two adjacent reactor containers.

17. Photobioreactor according to claim 1, wherein the overflow wall region has a peripheral frame with a container overflow opening surrounded by the frame, wherein it is provided that a frame part region which is lower in the vertical axis direction forms a connection region for the free end region of the front wall and the rear wall of the respectively associated reactor containers and that a frame part region which is upper in the vertical axis direction adjoins the top wall.

18. Photobioreactor according to claim 1, wherein the overflow wall region has at least one flow guide element projecting into the container overflow opening and several container overflow openings lying next to one another in the transverse direction.

19. Photobioreactor according to claim 17, wherein for the formation of several container overflow openings at least one connection web running between frame parts, at least one connection web running in the vertical axis direction and between frame parts opposite in the vertical axis direction, is provided as a flow guide element.

20. Photobioreactor according to claim 1, wherein the at least one lighting element is arranged in the gap between the adjacent reactor containers in such a way that regions of different brightness can be formed in the at least one reactor chamber of the adjacent reactor containers which is illuminated by the at least one lighting element, preferably providing regions of different brightness which lie one behind the other in the direction of flow can be formed.

21. Photobioreactor according to claim 1, wherein in the gap between the adjacent reactor containers a plurality of lighting elements are accommodated spaced apart from each other in the direction selected from the group consisting of the vertical axis direction and the transverse direction, wherein a plurality of transversely extending rows of lighting elements are provided which are spaced apart from each other in the vertical axis direction.

22. Photobioreactor according to claim 1, wherein the partition in the bottom wall side wall region has a peripheral frame region with a partition-through-flow opening surrounded by the frame region, wherein a lower frame part region in the vertical axis direction adjoins the bottom wall.

23. Photobioreactor according to claim 1, wherein the partition has at least one flow guide element projecting into the partition-through-flow opening and a plurality of partition-through-flow openings, lying next to one another in the transverse direction.

24. Photobioreactor according to claim 1, wherein at least one or at least part of the reactor containers has at least one feed nozzle as feeding device.

25. Photobioreactor according to claim 1, wherein an inlets for the nutrient medium is provided at the front reactor container in the longitudinal direction or flow direction in the wall selected from the group consisting of the top wall and the front wall and the side wall of the front reactor container in the longitudinal direction or flow direction, so that the nutrient medium can be supplied to the front reactor chamber of the front reactor container.

26. Photobioreactor according to claim 25, wherein the inlet is coupled to a conveying device simultaneously acting as a circulation device for the nutrient medium in the photobioreactor, by means of which a portion of the nutrient medium withdrawn from a rear region of the photobioreactor can be fed to the foremost reactor container.

27. Photobioreactor according to claim 1, wherein an outlet for the nutrient medium is provided at the rearmost reactor container in the longitudinal direction or flow direction, in the wall selected from the group consisting of the top wall and the rear wall and the side wall of the rearmost reactor container in the longitudinal direction or flow direction, so that the nutrient medium can be discharged from the rear reactor chamber of the rearmost reactor container.

28. Photobioreactor according to claim 1, wherein all reactor containers have an identical U-shaped basic structure with a front wall and a rear wall of substantially the same height, both of which have a gap distance to the top wall and both of which are surmounted by the partition extending up to a d adjoining the top wall;
wherein the gap distance to the top wall in the adjacent region of two reactor containers is bridged by the overflow wall region which extends to the top wall and is adjacent thereto;
wherein the front wall of the foremost reactor container in longitudinal direction or flow direction has a first wall and plate-like bridging element which extends up to and adjoins the top wall;
wherein the rear wall of the rearmost reactor container in the longitudinal direction or flow direction has a second wall-like and plate-like bridging element which extends to the top wall and is adjacent thereto; and
wherein the first and second wall and plate-like bridging element as well as all existing front walls, partitions and rear walls as well as the at least one overflow wall region extend in trans verse direction between the side walls likewise extending up to the top wall and adjoin there, so that a closed reactor is formed when the top wall mounted.

29. Biogas plant with at least one photobioreactor according to claim 1.

30. Reactor for gasification of fuels, in particular for gasification of carbonaceous solid fuels, with at least one photobioreactor according to claim 1.

31. Reactor container for a photobioreactor according to claim 1, wherein the reactor container is an upwardly open container which has a U-shape cross section with a preferably rectangular or plate-shaped front extending in the direction of the vertical axis and a preferably rectangular or plate-shaped rear wall spaced apart therefrom in the longitudinal direction and likewise extending in the direction of the vertical axis, which rear walls are connected to one another at the bottom by a bottom wall,
wherein a partition, is provided in the reactor container and extends upwards in the vertical axis direction starting from the bottom wall, so that the partition subdivides the reactor container, in relation to the longitudinal direction, into a front reactor chamber and a rear reactor chamber;
wherein in the partition, in the adjoining and connecting region adjoining and connecting region of the partition near the bottom wall, at least one partition-through-flow opening is provided between the front and the rear reactor chamber,
wherein the reactor container has at least one feeding device, by means of which a CO2-containing medium can be introduced from outside the reactor container; and
wherein the CO2-containing medium is a CO2-containing gas or CO2 derived from a CO2-containing gas.

32. Photobioreactor according to claim 1, wherein the photobioreactor is a closed reactor comprising a plurality of upwardly open reactor containers which are closed by a single-part or multi-part, top wall of the photobioreactor and in which a nutrient medium can be accommodated, wherein at least a part of the reactor containers is an individual container that has a U-shaped cross section with a front wall extending in the direction of the vertical axis and a rear wall spaced apart therefrom in the longitudinal direction and likewise extending in the direction of the vertical axis, which rear walls are connected to one another at the bottom by a bottom wall;
wherein the reactor containers of the photobioreactor, which are individual containers, are arranged one behind the other in the longitudinal direction in such a way that a front reactor container, adjoins a front wall, which is at least partially transparent to light, of a rear reactor container, forming a gap wherein the free end regions of the front and rear walls, which adjoin one another with the formation of the gap, have a common overflow wall region which closes the gap from above with respect to the vertical axis direction and which has at least one container overflow opening between the adjoining reactor containers
wherein the overflow wall region extends up to and adjoins the top wall, adjoining the top wall in a gas-tight and liquid-tight manner;
wherein at least one lighting element is accommodated in the gap between adjacent reactor containers, by means of which light element light can be emitted through the respectively associated front or rear wall, which is of light-permeable design at least in regions, into one of the two adjacent reactor containers or into both adjacent reactor containers;
wherein in each of the reactor containers designed as individual containers, a partition is provided, which, starting from the bottom wall, extends upwards in the vertical axis direction to the top wall acid adjoins the latter, preferably adjoins the top wall in a gas-tight and liquid-tight manner, so that the partition subdivides the reactor container with respect to the longitudinal direction, into a front reactor chamber and a rear reactor chamber;
wherein in the partition, in the bottom wall side adjoining region of the partition to the bottom wall, at least one partition-through-flow opening is provided between the front and the rear reactor chamber so that a nutrient medium received in the front reactor chamber of a front reactor container flows through the at least one partition-through-flow opening into the rear reactor chamber of the front reactor container and further flows from the rear reactor chamber of the front reactor container through the at least one container overflow opening into a front reactor chamber of a rear reactor container,
wherein at least a part of the reactor containers has at least one feeding device, by means of which a CO2-containing medium can be introduced from outside the reactor container into at least one reactor container; and
wherein the CO2-containing medium is a CO2-containing gas or CO2 derived from a CO2-containing gas.
